SEQUENCE LISTING (14 sequences)

SEQ ID NO: 1 - DNA, 2422 bp, Arabidopsis thaliana
atgaataatcagaagaagttgatgctattctcttatttttgcgccatttggtaaaacatttgaaattagtttcgaaatgcatgcgattttctgtcgcagggtcgccgtcggttgctcttccccgcagctaaccaaattggtgacgaagcaggcgagccagagtcgccattccctgagccatcttcttccttttgatctctcttcccgctttgttcctccttatgttgtatcacgatcggcgagagttcatggattcttcgcagggaagttgggaaacaccaatctgaagctgaaattcggaaatgttatggaatctagagctgggttcttcagttctgagcttcctagtcatggattcgagtctggtggttttactgggtttcaaaaacgtggatggtatcgtttcttttcctctccctttttgtttttcaaagtaaagttggaacctttcctaagaaaaattctgagttcccctttctctatggtcctctgttttatattgtacctatttgtttccaagtaaaaactacatttttcaaagataaatttgtcttcaccataaaatttctgggtttgacaggatttatttggtaaagcatcgaccttttttcacaactctctttagcttttctggtcaattgtacgtttggctcttccaagtaaaagacgtgtcaggattcagagaaattagtaggctttttttttcttccatttctgcgtagttcttgattttgagcagcacaaagctcttttgatgcttttctctttatagataagacatagaatagcaagctggacactgtttagaccttctcctttaaaaaattgttctcctacatcaaaaatcttagattctttacctgctccatttgattttggatactaataccaaaaagaaaaaaccactttattggtttaactctggcaggaagtcttggattaatggagctaatggtgtagtttttggattggtaatagctaatgctgctgtatttacaatgtggcgggttttaggcaaggacaatatgtggatggtgaagaattttatggtctgacctcatcaaattctaacaacttttttaaaaataaaaaatctacatgtattattaaccatatggtatttggacagttatcaaggtacagttttatgactggacgtatacacacgctgatcacttcgggtttcagtcatgttggagccactcatatcatcttgaatatgatggggctttgctacttcggagccagagtaagattttttccttgtttccatatattaattttcgtaaacgaaatgagaaatgctttcatttgtttcctatgaagtatgaaccggctacttacacttaatctggctcatctaaggaccgtccttagttcgtttaaaaagaaaaacttgctacctattgatctttatatcgaatcccatttgttcttagattgcaagaagctttgggccgcgctaccttttgaagctctactttgctggagcacttggtggctctgttttcttcttgagtagtcatgctctctcggttatatcactcaaggtatagtgcaacaagcataattttctcattttcctaaccatgctaggtccacagatgtgtagtttttccagtgcatttgtactacgtacatgttctctatgtgtttgtgaggtttagacgaaatgctttacctttgttattggttaatcaatcatacagggccaaagagttgtccctaaggatcaattaaaagtccccattgggaagctggtatgttttttcactcatccagttaaattattttttaaaagtataaacagactggaacagagatgcacacgctcatgttttggatggtcaaactctttccaatttgtgtacagggtgctaatggacctgtgtatgccattacgcttctcgatatgctcctttacccaaaagttactacatactttgggttgatgctccgagtgcctgtatttgctgtaaggtctctctagatactcgtcataatgagtgaaacgtttctgaatcatctaaaatactctcactctcttaatcttcagggaatctatagcttaggactcaacattataaagatgctagaggtgtgtatgatgtgagaatctaatccacatttgctttgagactatatgactatatcttaaaccagaccaacttttttgggaatgcaggggaaaaacaacaataccttgactagtttggatcagttgggaggagttgtagttgcagtcattgcgtgggcaaggataaggaaaggtcgattctgctactgattgattattttactttctccatcaaaccttggacttgctgcaaaagattcatataacttgtcccatcattatactactggagtactggttatgttaaactttgtgacaatgagattattattattactatttttt

SEQ ID NO: 2 - DNA, 1180 bp, Arabidopsis thaliana
atgaataatcagaagaagttgatgctattctcttatttttgcgccatttggtaaaacatttgaaattagtttcgaaatgcatgcgattttctgtcgcagggtcgccgtcggttgctcttccccgcagctaaccaaattggtgacgaagcaggcgagccagagtcgccattccctgagccatcttcttccttttgatctctcttcccgctttgttcctccttatgttgtatcacgatcggcgagagttcatggattcttcgcagggaagttgggaaacaccaatctgaagctgaaattcggaaatgttatggaatctagagctgggttcttcagttctgagcttcctagtcatggattcgagtctggtggttttactgggtttcaaaaacgtggatggaagtcttggattaatggagctaatggtgtagtttttggattggtaatagctaatgctgctgtatttacaatgtggcgggttttaggcaaggacaatatgtggatggtgaagaattttatgttatcaaggtacagttttatgactggacgtatacacacgctgatcacttcgggtttcagtcatgttggagccactcatatcatcttgaatatgatggggctttgctacttcggagccagaattgcaagaagctttgggccgcgctaccttttgaagctctactttgctggagcacttggtggctctgttttcttcttgagtagtcatgctctctcggttatatcactcaagggccaaagagttgtccctaaggatcaattaaaagtccccattgggaagctgggtgctaatggacctgtgtatgccattacgcttctcgatatgctcctttacccaaaagttactacatactttgggttgatgctccgagtgcctgtatttgctggaatctatagcttaggactcaacattataaagatgctagaggggaaaaacaacaataccttgactagtttggatcagttgggaggagttgtagttgcagtcattgcgtgggcaaggataaggaaaggtcgattctgctactgattgattattttactttctccatcaaaccttggacttgctgcaaaagattcatataacttgtcccatcattatactactggagtactggttatgttaaactttgtgacaatgagattattattattactatttttt

SEQ ID NO: 3 - DNA, 1191 bp, Arabidopsis thaliana
atgaataatcagaagaagttgatgctattctcttatttttgcgccatttggtaaaacatttgaaattagtttcgaaatgcatgcgattttctgtcgcagggtcgccgtcggttgctcttccccgcagctaaccaaattggtgacgaagcaggcgagccagagtcgccattccctgagccatcttcttccttttgatctctcttcccgctttgttcctccttatgttgtatcacgatcggcgagagttcatggattcttcgcagggaagttgggaaacaccaatctgaagctgaaattcggaaatgttatggaatctagagctgggttcttcagttctgagcttcctagtcatggattcgagtctggtggttttactgggtttcaaaaacgtggatggaagtcttggattaatggagctaatggtgtagtttttggattggtaatagctaatgctgctgtatttacaatgtggcgggttttaggcaaggacaatatgtggatggtgaagaattttatgttatcaaggtacagttttatgactggacgtatacacacgctgatcacttcgggtttcagtcatgttggagccactcatatcatcttgaatatgatggggctttgctacttcggagccagaattgcaagaagctttgggccgcgctaccttttgaagctctactttgctggagcacttggtggctctgttttcttcttgagtagtcatgctctctcggttatatcactcaagggccaaagagttgtccctaaggatcaattaaaagtccccattgggaagctgggtgctaatggacctgtgtatgccattacgcttctcgatatgctcctttacccaaaagttactacatactttgggttgatgctccgagtgcctgtatttgctggaatctatagcttaggactcaacattataaagatgctagaggtgtgtatgatgggaaaaacaacaataccttgactagtttggatcagttgggaggagttgtagttgcagtcattgcgtgggcaaggataaggaaaggtcgattctgctactgattgattattttactttctccatcaaaccttggacttgctgcaaaagattcatataacttgtcccatcattatactactggagtactggttatgttaaactttgtgacaatgagattattattattactatttttt

SEQ ID NO: 4 - DNA, 1185 bp, Arabidopsis thaliana
atgaataatcagaagaagttgatgctattctcttatttttgcgccatttggtaaaacatttgaaattagtttcgaaatgcatgcgattttctgtcgcagggtcgccgtcggttgctcttccccgcagctaaccaaattggtgacgaagcaggcgagccagagtcgccattccctgagccatcttcttccttttgatctctcttcccgctttgttcctccttatgttgtatcacgatcggcgagagttcatggattcttcgcagggaagttgggaaacaccaatctgaagctgaaattcggaaatgttatggaatctagagctgggttcttcagttctgagcttcctagtcatggattcgagtctggtggttttactgggtttcaaaaacgtggatggaagtcttggattaatggagctaatggtgtagtttttggattggtaatagctaatgctgctgtatttacaatgtggcgggttttaggcaaggacaatatgtggatggtgaagaattttatgttatcaaggtacagttttatgactggacgtatacacacgctgatcacttcgggtttcagtcatgttggagccactcatatcatcttgaatatgatggggctttgctacttcggagccagaattgcaagaagctttgggccgcgctaccttttgaagctctactttgctggagcacttggtggctctgttttcttcttgagtagtcatgctctctcggttatatcactcaagggccaaagagttgtccctaaggatcaattaaaagtccccattgggaagctgggtgctaatggacctgtgtatgccattacgcttctcgatatgctcctttacccaaaagttactacatactttgggttgatgctccgagtgcctgtatttgctgtaagggaatctatagcttaggactcaacattataaagatgctagaggggaaaaacaacaataccttgactagtttggatcagttgggaggagttgtagttgcagtcattgcgtgggcaaggataaggaaaggtcgattctgctactgattgattattttactttctccatcaaaccttggacttgctgcaaaagattcatataacttgtcccatcattatactactggagtactggttatgttaaactttgtgacaatgagattattattattactatttttt

SEQ ID NO: 5 - DNA, 1977 bp, Arabidopsis thaliana
ctaaaataaaaggtccatttgttgggaaattagaaacgtgaaggggaagatggtatcagtgtcattatctcatcataatctctggccaccggaatctggatctacggcgtttcgtggtttcgcaaccgccgcttccgtacacgcctgccacatgttagccgtcacctccgccttgatttccatcttcgctcttcccttaaggttcacctactcatcttcgctcgtttgtagtataattcctggaatcacaaagcttaaattgcttcgtcaggcgcttctgatttctgggttaatctcattttatttgactatgctctgcttctacagaaactgcagcatttttctgatgatgcaagaatgaagtttgcaagataccaaagagtatgtattcaatggggctaattttctcaagtctagagttgatatccggttgtcccaatcttcgccatttgtatgtttctttaatggtggagagagtagattaaaccctagaggtggtgaggaaggatcatcgaacccggagacttccaagagaaacacagttaacggacggagatggaccaatgtccttcttgccattaacgttatgtatgtgttctgttctctattacatgaaaaagattatgtcttttggtttggtatgtgttgcattagaagaatgcttttataatcaagcttgagacttgagagttgtgagctaagaaggcttatggatttcattttgtgtttatgatgctagaatgtatattgcacaaattgcatccgatgaaggtcctgacatggggagccaaggtactgtagacttagttaaatgagaggcagttttttcttgctgcaaatttgatgtatgtggatgattgcagataaacagtttaatcgaaagaggtcagctatggagactggctacagcttctgttcttcatgcgaatcctatgcatctcatggtctgtttctaaccaatccttcaccctttgtaatatctaatcattgctctgtatgctaaactgtttctcctttgttttagataaattgctattctttgaattctattggaccaactgctgagagtttaggtggcccaaagagatttcttgctgtttacttgacatccgcagttgcaagtaattgacgactcatagactttctttgatcttgatgcttatatatctctgtttcgagtttctgactcacagaacccatcttaagagttctaggttcagcaatgagctactggttcaacaaagcgccatcagttggtgcttcaggtgcaatattcggattggtaagtctcgtgtagccataagcctttgttttagacacgtctcaaagactcatacaaatgaagttctttaaacgaatgtcccttttgtttgagtaggttggctcggtagctgtatttgttataagacacaaacagatggtcagaggtggcaatgaggatctaatgcagatagctcaaattattgccctaaacatggtaagtaatacaacagagaaaattgtataatctctcttaagttctaacaagcttttatcactatgcaggcaatgggtctaatgtccagacgtatagataactggggacatgtaagtaacccaattatgccttcaaatgtattaaccagtctccgattttttctcggatcagtggtctgataacatctccggtgaattcagattggtggcttacttggtggaaccgcaatgacatggcttcttggaccacagtggaagtatgaatatacaacaagagacggtcggagagtcttcatggacagtgctcctattcctctccttttgcggtggagaaatgaacagcgacggctttgattcaagaccaacgaagctgaacattttctctctcaatgccgtgtgaatataggaaatgaatgtatcttgaaagattcgatccttgatcaaagtagtagcttattgtattacagaaatgcaattgtactactaaataattgttgtttt

SEQ ID NO: 6 - DNA, 815 bp, Arabidopsis thaliana
tgtcattatctcatcataatctctggccaccggaatctggatctacggcgtttcgtggtttcgcaaccgccgcttccgtacacgcctgccaccatgttagccgtcacctccgccttgatttccatcttcgctcttcccttaagaaactgcagcatttttctgatgatgcaagaatgaagtttgcaagataccaaagagtatttgtattcaatggggctaattttctcaagtctagagttgatatccggttgtcccaatcttcgccatttgtatgtttctttaatggtggagagagtagattaaaccctagaggtggtgaggaaggatcatcgaacccggagacttccaagagaaacacagttaacggacggagatggaccaatgtccttcttgccattaacgttataatgtatattgcacaaattgcatccgatgggaaggtcctgacatggggagccaagataaacagtttaatcgaaagaggtcagctatggagactggctacagcttctgttcttcatgcgaatcctatgcatctcatgataaattgctattctttgaattctattggaccaactgctgagagtttaggtggcccaaagagatttcttgctgtttacttgacatccgcagttgcaaagttctaggttcagcaatgagctactggttcaacaaagcgccatcagttggtgcttcaggtgcaatattcggattggttggctcggtagctgtatttgttataagacacaaacagatggtcagaggtggcaatgaggatctaatgcagatagctcaaattattgccctaaacatgg

SEQ ID NO: 7 - DNA, 807 bp, Arabidopsis thaliana
tgtcattatctcatcataatctctggccaccggaatctggatctacggcgtttcgtggtttcgcaaccgccgcttccgtacacgcctgccaccatgttagccgtcacctccgccttgatttccatcttcgctcttcccttaagaaactgcagcatttttctgatgatgcaagaatgaagtttgcaagataccaaagagtatttgtattcaatggggctaattttctcaagtctagagttgatatccggttgtcccaatcttcgccatttgtatgtttctttaatggtggagagagtagattaaaccctagaggtggtgaggaaggatcatcgaacccggagacttccaagagaaacacagttaacggacggagatggaccaatgtccttcttgccattaacgttataatgtatattgcacaaattgcatccgatgggaaggtcctgacatggggagccaagataaacagtttaatcgaaagaggtcagctatggagactggctacagcttctgttcttcatgcgaatcctatgcatctcatgataaattgctattctttgaattctattggaccaactgctgagagtttaggtggcccaaagagatttcttgctgtttacttgacatccgcagttgcaagttcagcaatgagctactggttcaacaaagcgccatcagttggtgcttcaggtgcaatattcggattggttggctcggtagctgtatttgttataagacacaaacagatggtcagaggtggcaatgaggatctaatgcagatagctcaaattattgccctaaacatgg

SEQ ID NO: 8 - DNA, 696 bp, Homo sapiens
atgcagtatttctttggcatcactgcagctagtaatttgccttctggattcctggcacctgtgtttgctctgtttgtaccattttactgctccataccaagagtccaagtggcacaaattctgggtccgttgtccatcacaaacaagacattgatttatatattgggactgcagcttttcacctctggttcctacatctggattgtagccataagtggacttatgtccggtctgtgctacgacagcaaaatgttccaggtgcatcaggtgctctgcatccccagctggatggcaaaattcttttcttggacacttgaacccatcttctcttcttcagaacccaccagcgaagccagaattgggatgggagccacgctggacatccagagacagcagagaatggagctgctggaccggcagctgatgttctctcagtttgcacaagggaggcgacagagacagcagcagggaggaatgatcaattggaatcgtctttttcctcctttacgtcagcgacaaaacgtaaactatcagggcggtcggcagtctgagccagcagcgccccctctagaagtttctgaggaacaggtcgcccggctcatggagatgggattttccagaggtgatgctttggaagccctgagagcttcaaacaatgacctcaatgtcgccaccaacttcctgctgcagcactga

SEQ ID NO: 9 - protein, 322 aa, Arabidopsis thaliana
MHAIFCRRVAVGCSSPQLTKLVTKQASQSRHSLSHLLPFDLSSRFVPPYVVSRSARVHGFFAGKLGNTNLKLKFGNVMESRAGFFSSELPSHGFESGGFTGFQKRGWKSWINGANGVVFGLVIANAAVFTMWRVLGKDNMWMVKNFMLSRYSFMTGRIHTLITSGFSHVGATHIILNMMGLCYFGARIARSFGPRYLLKLYFAGALGGSVFFLSSHALSVISLKGQRVVPKDQLKVPIGKLGANGPVYAITLLDMLLYPKVTTYFGLMLRVPVFAGIYSLGLNIIKMLEGKNNNTLTSLDQLGGVVVAVIAWARIRKGRFCY

SEQ ID NO: 10 - protein, 299 aa, Arabidopsis thaliana
MHAIFCRRVAVGCSSPQLTKLVTKQASQSRHSLSHLLPFDLSSRFVPPYVVSRSARVHGFFAGKLGNTNLKLKFGNVMESRAGFFSSELPSHGFESGGFTGFQKRGWKSWINGANGVVFGLVIANAAVFTMWRVLGKDNMWMVKNFMLSRYSFMTGRIHTLITSGFSHVGATHIILNMMGLCYFGARIARSFGPRYLLKLYFAGALGGSVFFLSSHALSVISLKGQRVVPKDQLKVPIGKLGANGPVYAITLLDMLLYPKVTTYFGLMLRVPVFAGIYSLGLNIIKMLEVCMMGKTTIP

SEQ ID NO: 11 - protein, 281 aa, Arabidopsis thaliana
MHAIFCRRVAVGCSSPQLTKLVTKQASQSRHSLSHLLPFDLSSRFVPPYVVSRSARVHGFFAGKLGNTNLKLKFGNVMESRAGFFSSELPSHGFESGGFTGFQKRGWKSWINGANGVVFGLVIANAAVFTMWRVLGKDNMWMVKNFMLSRYSFMTGRIHTLITSGFSHVGATHIILNMMGLCYFGARIARSFGPRYLLKLYFAGALGGSVFFLSSHALSVISLKGQRVVPKDQLKVPIGKLGANGPVYAITLLDMLLYPKVTTYFGLMLRVPVFAVRESIA

SEQ ID NO: 12 - protein, 283 aa, Arabidopsis thaliana
MVSVSLSHHNLWPPESGSTAFRGFATAASVHACHHVSRHLRLDFHLRSSLKKLQHFSDDARMKFARYQRVFVFNGANFLKSRVDIRLSQSSPFVCFFNGGESRLNPRGGEEGSSNPETSKRNTVNGRRWTNVLLAINVIMYIAQIASDGKVLTWGAKINSLIERGQLWRLATASVLHANPMHLMINCYSLNSIGPTAESLGGPKRFLAVYLTSAVARVLGSAMSYWFNKAPSVGASGAIFGLVGSVAVFVIRHKQMVRGGNEDLMQIAQIIALNMAMGLMSRR

SEQ ID NO: 13 - protein, 280 aa, Arabidopsis thaliana
MVSVSLSHHNLWPPESGSTAFRGFATAASVHACHHVSRHLRLDFHLRSSLKKLQHFSDDARMKFARYQRVFVFNGANFLKSRVDIRLSQSSPFVCFFNGGESRLNPRGGEEGSSNPETSKRNTVNGRRWTNVLLAINVIMYIAQIASDGKVLTWGAKINSLIERGQLWRLATASVLHANPMHLMINCYSLNSIGPTAESLGGPKRFLAVYLTSAVASSAMSYWFNKAPSVGASGAIFGLVGSVAVFVIRHKQMVRGGNEDLMQIAQIIALNMAMGLMSRR

SEQ ID NO: 14 - protein, 231 aa, Homo sapiens
MQYFFGITAASNLPSGFLAPVFALFVPFYCSIPRVQVAQILGPLSITNKTLIYILGLQLFTSGSYIWIVAISGLMSGLCYDSKMFQVHQVLCIPSWMAKFFSWTLEPIFSSSEPTSEARIGMGATLDIQRQQRMELLDRQLMFSQFAQGRRQRQQQGGMINWNRLFPPLRQRQNVNYQGGRQSEPAAPPLEVSEEQVARLMEMGFSRGDALEALRASNNDLNVATNFLLQH

(19) United States
(12) Patent Application Publication
(10) Pub. No.: US 2016/0129078 A1
(43) Pub. Date: May 12, 2016

(54) Rhomboid Proteins and Uses Thereof

(71) Applicant: Queen's University at Kingston, Kingston (CA)

(72) Inventors: Kenton Ko, Kingston (CA); Joshua Powles, Kingston (CA)

(21) Appl. No.: 14/879,712

(22) Filed: Oct. 9, 2015

Related U.S. Application Data
(60) Provisional application No. 62/062,470, filed on Oct. 10, 2014.

(51) Int. Cl. A61K 38/16; A61K 31/517; A61K 31/65; A61K 31/43; A61K 31/7048

(52) U.S. Cl. CPC A61K 38/168; A61K 31/43; A61K 31/7048; A61K 31/65; A61K 31/517

(57) ABSTRACT

Compositions and methods for managing the growth, life cycle, functionality, or biological progression of cells, tissues, and organisms by introducing rhomboid proteins or peptides into the internal and external environment of the cells, tissues, and organisms. In particular, embodiments comprise compositions and methods for overcoming drug resistance in disease, and for sensitizing cells, tissues, and organisms to agents to treat disease, and methods for producing such compositions. The compositions and methods include rhomboid proteins, polypeptides, and/or peptides, and nucleic acids encoding such proteins, polypeptides, and/or and peptides, corresponding to the Arabidopsis At1g74130 or At1g25290 gene or human UBAC2 gene. The rhomboid proteins, polypeptides, and/or and peptides or nucleic acids may be derived from natural genetic sources, from different species, different genes of the rhomboid family, through alternative splicing of encoding transcripts, through laboratory manipulations, using recombinant protein production techniques, fermentation, mutagenesis approaches, synthesis, peptides, or other methods of generation and production.

### RELATED APPLICATION

This application claims the benefit of the filing date of U.S. Application No. 62/062,470, filed on Oct. 10, 2014, the contents of which are incorporated herein by reference.

### BACKGROUND

Genes encoding rhomboid proteases and proteins appear in the genomes of diverse organisms, from bacteria to human. Rhomboid genes can be divided into sub-groups encoding proteolytically active secretase- and presenilins-associated rhomboid-like (PARL)-type rhomboids, and two sub-groups of catalytically inactive forms such as iRhoms, derlins, and other distantly related forms.

The rhomboid proteins categorized as secretase-like or PARL-like appear to be mostly active proteases and are frequently observed to possess roles in regulation, often involving interacting and cleaving specific membrane-residing substrate targets. This has been observed in a wide range of cellular activities such as in the epidermal growth factor (EGF) signaling pathway of Drosophila melanogaster, in the intercellular quorum-sensing mechanism of the bacterium Providencia stuartii, and in mitochondrial membrane remodeling of the baker's yeast, Saccharomyces cerevisiae.

The two sub-groups of inactive rhomboid-like proteins are believed to lack the essential catalytic residues utilized by active rhomboid proteases. These inactive rhomboid-like proteins have nevertheless been observed to be biologically functional even without the proteolytic capabilities typically associated with the active rhomboid proteases. Four such cases have been reported to date. The human iRhom1 (p100hRho/RHBDF1) was found to interact with TGF-alpha-like ligands, required for epithelial cancer cell survival, and involved with GPCR-mediated transactivation of EGFR growth signals in head and squamous cancer cells. In Drosophila, the inactive iRhom Rhomboid-5 was found to be a regulator of intercellular signaling, not by cleaving rhomboid substrates, but by preventing cleavage during the quality control mechanism of the ER. In mammalian ER, inactive derlins (pseudoproteases) were found to be involved in the protein dislocation. In Arabidopsis, the inactive At1g74130 rhomboid protein appeared to play a role during the early stages of tissue development and organelle biogenesis. Plants lacking At1g74130 displayed a short delay in development and organelle biogenesis when grown naturally without the stress of transplantation.

Alternative splicing is a mechanism for generating additional protein forms that may bear functionality. In the case of the human RHBDD2 (rhomboid domain containing 2) gene, two alternatively spliced mRNA isoforms were found to be expressed at elevated levels in breast cancer biopsies and cell lines. The roles of the two splice biotypes remain to be elucidated.

Although a small number of natural rhomboid protease substrates (hence their corresponding processes) have been revealed to date, in organisms including insects, mammals, and plants, the roles of rhomboid proteases and proteins remain to be elucidated in many cases. Equally unknown are their modes of operation, for example, it is not known if rhomboid proteases or proteins work independently, singularly, or as complexes of same or different rhomboid proteins, or whether such processes or complexes are modulated in response to a given situation.

### SUMMARY

According to a broad aspect, described herein are compositions and methods for managing the growth, life cycle, functionality, developmental processes, or biological progression of cells, tissues, and organisms by introducing rhomboid proteins into the environment of the cells, tissues, and organisms. The modes of effect or function can be either antagonistic or agonistic. In particular, embodiments comprise compositions and methods for overcoming drug resistance in disease, and for sensitizing cells, tissues, and organisms to agents to treat disease, and methods for producing such compositions.

The compositions and methods may include active or inactive rhomboid proteins, polypeptides, and/or peptides, and nucleic acids encoding such proteins and peptides. In some embodiments, the rhomboid proteins, polypeptides, and/or peptides, and nucleic acids correspond to the Arabidopsis At1g74130 gene. The inactive rhomboid proteins, polypeptides, and peptides or nucleic acids encoding such proteins and peptides can be derived from natural genetic sources, from different species, or different genes of the rhomboid family, through alternative splicing of encoding transcripts, through laboratory manipulations, using recombinant protein production techniques, fermentation, mutagenesis approaches, synthesis, peptides, or other methods of generation and production. For example, nucleic acids which hybridize to the At1g74130 gene, or a functional equivalent, may also be used when such hybridizing or aligning sequences encode a functional equivalent of the At1g74130 protein, polypeptides, or peptides.

The compositions and methods may be used in applications such as management of yeast cell growth or functionality or infections in combination with anti-fungals to enhance treatment efficacy; management of bacterial cell growth or infections in combination with antibiotics to enhance treatment efficacy, and management of malarial life cycle progression or infections in combination with anti-malarials to enhance treatment efficacy. However, as described below, the compositions and methods are not limited thereto.

Features of the compositions and methods include one or more of: effective for a broad range of organisms and biological systems; applicable to live cells; comprising a protein or its encoding nucleic acid sequence; effective for broad range of targets; not limited by specific drugs; activity based on protein interactions; resistant to mutative pressure; and strength can be manipulated easily.

Cellular systems to which the compositions and methods may be applied can be those of prokaryotic or eukaryotic cells or variations of the two categories, unicellular or multicellular. They can include the whole cell, any external or internal compartment or structure, any organellar membrane, any location in the membrane or side of the membrane, either single- or double-membrane systems, as well as the plasma membrane.

One embodiment relates to a method for altering translocation and/or expression of the products of bacterial fermentation or culture or in natural contexts. The nucleic acids and/or amino acids described herein can be used to facilitate and alter the synthesis, secretion, cleavage, processing, distribution, and/or release of products as a result of the impact exerted when encoded products of such nucleic acids and/or amino acid products are incorporated into bacteria or associated with the cellular membranes of bacteria.

In another embodiment, nucleic acids and/or amino acids may be used to alter the growth or functionality of nonhuman organisms, and to produce different quantities of selected substances. These substances may include proteins and other molecules which are translocated, integrated, distributed, cleaved, or processed by cells, as well as substances which are incorporated into cell membranes of all types: i.e., plasma membranes, plastid membranes (including thylakoids), mitochondrial membranes (including cristae), Golgi membranes, endoplasmic reticula membranes, biofilms, extra cellular matrix, and the like.

Another embodiment relates to providing a vector comprising the DNA of SEQ ID NO:1, SEQ ID NO:2, SEQ ID NO:3, or SEQ ID NO:4, or a nucleic acid sequence which hybridizes or aligns thereto, and a promoter, which vector encodes rhomboid protein At1g74130 (SEQ ID NO:5), or a variant or a functional equivalent, such as SEQ ID NO:6 or SEQ ID NO:7, including any hybridizing or aligning nucleic acid sequence capable of directing alterations or variations in a manner similar to At1g74130.

Such vectors can be used in host cells to alter functions and processes that occur in cellular membranes. In addition, such vectors can be incorporated into the cells of multicellular organisms to alter functions and processes that occur in plasma membranes and/or organelle membranes.

Other embodiments provide modification of the functional capabilities of plastidial or mitochondrial compartments. Modifications may alter the accumulation of protein products in plastids or mitochondria, or the importation of enzymes involved in various biochemical or physiological pathways that function within the plastid or mitochondrion. Such alterations may impact the amount of biochemical or physiological activity in plastids or mitochondria where, for example, valuable products or functions may be created using the compositions and methods described herein. Such alterations may be used to increase or decrease product synthesis. For example, such modifications may be carried out bacteria so that products based on bacterial secretion, exportation, integration, or the like, are altered. Impact on organelles, such as mitochondria, may also alter cellular processes like apoptosis.

Other embodiments provide compositions and methods for modifying (e.g., enhancing) efficacy of an agent provided to a cell, tissue, or organism, or modifying (e.g., enhancing) transport of a substance across or into a cellular membrane, comprising: a) providing to the cell, tissue, or organism the agent or substance and (i) a protein comprising SEQ ID NO:5, SEQ ID NO:6, or SEQ ID NO:7; or (ii) polypeptide comprising at least a functional amino acid sequence corresponding to a portion of SEQ ID NO:5; wherein efficacy of the agent or transport of the substance is modified (e.g., enhanced). For example, a functional amino acid sequence may comprise 17 consecutive amino acid residues between residues 43 and 323 of SEQ ID NO:5.

Other embodiments provide compositions and methods for modifying (e.g., enhancing) efficacy of an agent provided to a cell, tissue, or organism, or modifying (e.g., enhancing) transport of a substance across or into a cellular membrane, comprising: a) providing to the cell, tissue, or organism the agent or substance and (i) a fusion protein comprising SEQ ID NO:5, SEQ ID NO:6, or SEQ ID NO:7 or its peptides and another proteinaceous component such as an antibody, an immunoglobulin, a targeting signal, or another membrane protein or peptide; or (ii) a fusion polypeptide comprising at least 17 consecutive amino acid residues of SEQ ID NO:5; and another proteinaceous component such as an antibody, an immunoglobulin, a targeting signal, or another membrane protein or peptide; wherein efficacy of the agent or transport of the substance is modified.

One embodiment provides a method for enhancing efficacy of an agent, comprising: co-administering to a cell, tissue, organ, or organism the agent in a therapeutic or sub-therapeutic dose and a rhomboid protein, polypeptide, or peptide, in a suitable vehicle; wherein the rhomboid protein is Arabidopsis At1g74130 protein or a splice variant thereof, or the polypeptide or peptide is derived from Arabidopsis At1g74130 protein or a splice variant thereof, or is a functional equivalent thereof; wherein said co-administration enhances efficacy of the agent in the cell, tissue, organ, or organism.

The methods and compositions may comprise two or more agents; wherein each agent has a different biological activity or function. At least one agent may be a therapeutic agent.

Co-administering or providing may comprise administering the agent and the rhomboid protein, polypeptide, or peptide together (i.e., simultaneously or substantially simultaneously), or sequentially (i.e., one followed by the other after a selected period of time). Further, co-administering may comprise administering the two or more agents together (i.e., simultaneously or substantially simultaneously), or sequentially (i.e., one followed by the other after a selected period of time).

Enhancing efficacy of the agent may include treating a disease with the agent, reducing resistance to the agent in the cell, tissue, or organism, or sensitizing the cell, tissue, or organism to the agent.

Another embodiment provides a method for enhancing transport of an agent across or into a cellular membrane, comprising: providing to a cell, tissue, organ, or organism the agent and a rhomboid protein, polypeptide, or peptide, in a suitable vehicle; wherein the rhomboid protein is Arabidopsis At1g74130 protein or a splice variant thereof, or the polypeptide or peptide is derived from Arabidopsis At1g74130 protein or a splice variant thereof, or is a functional equivalent thereof; wherein the rhomboid protein or peptide enhances transport of the agent across or into the cellular membrane in the cell, tissue, organ, or organism.

Another embodiment provides a composition, comprising: at least one agent and a rhomboid protein, polypeptide, or peptide, with a suitable vehicle; wherein the rhomboid protein is Arabidopsis At1g74130 protein or a splice variant thereof, or the polypeptide or peptide is derived from Arabidopsis At1g74130 protein or a splice variant thereof, or is a functional equivalent thereof.

Another embodiment provides a composition, comprising: a rhomboid protein, polypeptide, or peptide as a fusion with a suitable vehicle; wherein the rhomboid protein is Arabidopsis At1g74130 protein or a splice variant thereof, or the polypeptide or peptide is derived from Arabidopsis At1g74130 protein or a splice variant thereof, or is a functional equivalent thereof.

Another embodiment provides a composition, comprising: a rhomboid protein, polypeptide, or peptide; wherein the rhomboid protein is Arabidopsis At1g74130 protein or a splice variant thereof, or the polypeptide or peptide is derived from Arabidopsis At1g74130 protein or a splice variant thereof, or is a functional equivalent thereof.

Another embodiment provides a method for enhancing efficacy of an agent, comprising: co-administering to a cell, tissue, organ, or organism the agent in a therapeutic or sub-therapeutic dose and a rhomboid protein, polypeptide, or peptide, in a suitable vehicle; wherein the rhomboid protein is selected from At1g74130, At1g25290, and UBAC2 protein or a splice variant thereof, or the rhomboid polypeptide or peptide is derived from At1g74130, At1g25290, or UBAC2 protein or a splice variant thereof, or is a functional equivalent thereof; wherein said co-administration enhances efficacy of the agent in the cell, tissue, organ, or organism.

The method may comprise co-administering two or more agents; wherein each agent has a different biological activity or function. The protein or polypeptide may comprise SEQ ID NO:9, SEQ ID NO:10, SEQ ID NO: 11, SEQ ID NO:12, SEQ ID NO:13, or SEQ ID NO:14, or a functional equivalent thereof. The method may comprise enhancing efficacy of the agent including treating a disease with the agent. The method may comprise enhancing efficacy of the agent, including reducing resistance to the agent in the cell, tissue, or organism. The method may comprise enhancing efficacy of the agent, including sensitizing the cell, tissue, or organism to the agent. The method may comprise enhancing efficacy of the agent, including reducing or inhibiting production of a biofilm by the cell, tissue, or organism.

Another embodiment provides a method for enhancing transport of an agent across or into a cellular membrane, comprising: providing to a cell, tissue, organ, or organism the agent and a rhomboid protein, polypeptide, or peptide, in a suitable vehicle; wherein the rhomboid protein is selected from At1g74130, At1g25290, and UBAC2 protein or a splice variant thereof, or the polypeptide or peptide is derived from At1g74130, At1g25290, or UBAC2 protein or a splice variant thereof, or is a functional equivalent thereof; wherein the rhomboid protein or peptide enhances transport of the agent across or into the cellular membrane in the cell, tissue, organ, or organism.

In various embodiments the protein or polypeptide may comprise SEQ ID NO:9, SEQ ID NO:10, SEQ ID NO: 11, SEQ ID NO:12, SEQ ID NO:13, or SEQ ID NO:14, or a functional equivalent thereof.

In various embodiments the method may comprise treating antibiotic or drug resistance, infections including parasitic and fungal infections, cancerous cell growth, and progression of disease including Alzheimer's, Parkinson's, and Type-2 Diabetes.

Also described herein is a composition, comprising at least one agent and a rhomboid protein, polypeptide, or peptide, with a suitable vehicle; wherein the rhomboid protein is selected from At1g74130, At1g25290, and UBAC2 protein or a splice variant thereof, or the polypeptide or peptide is derived from At1g74130, At1g25290, or UBAC2 protein or a splice variant thereof, or is a functional equivalent thereof.

In various embodiments the composition may include a protein or polypeptide comprising SEQ ID NO:9, SEQ ID NO:10, SEQ ID NO:11, SEQ ID NO:12, SEQ ID NO:13, or SEQ ID NO:14, or a functional equivalent thereof. In other embodiments the rhomboid protein may comprise an amino acid sequence with at least 80% sequence identity to SEQ ID NO:9, SEQ ID NO:10, SEQ ID NO:11, SEQ ID NO:12, SEQ ID NO:13, or SEQ ID NO:14.

The at least one agent may be a therapeutic agent. The rhomboid protein, polypeptide, or peptide may comprise a fusion with the suitable vehicle.

In various embodiments the composition may be used in treating antibiotic or drug resistance, infections including parasitic and fungal infections, cancerous cell growth, and progression of disease including Alzheimer's, Parkinson's, and Type-2 Diabetes. In one embodiment the composition is used as an antibiofilm agent.

The methods and compositions may be used for, but not limited to, treating antibiotic or drug resistance, treating infections including parasitic and fungal infections, impairing biofilm production by cells, impairing secretion of materials by cells, treating cancerous cell growth, and inhibiting or reducing progression of disease including cancer, Alzheimer's, Parkinson's, and Type-2 Diabetes.

### BRIEF DESCRIPTION OF THE FIGURES

For a greater understanding of the invention, and to show more clearly how it may be carried into effect, embodiments will be described, by way of example, with reference to the accompanying drawings, wherein:

FIG. 1A shows the nucleic acid sequence which encodes the At1g74130 protein (SEQ ID NO:1).

FIG. 1B shows the cDNA sequence for the L-form splice variant of the At1g74130 protein (SEQ ID NO:2).

FIG. 1C shows the cDNA sequence for the 7/8 M-form splice variant of the At1g74130 protein (SEQ ID NO:3).

FIG. 1D shows the cDNA sequence for the 6/7 S-form splice variant of the At1g74130 protein (SEQ ID NO:4).

FIG. 1E shows the nucleic acid sequence which encodes the At1g25290 protein (SEQ ID NO:5).

FIG. 1F shows the cDNA sequence for the L-form splice variant of the At1g25290 protein (SEQ ID NO:6).

FIG. 1G shows the cDNA sequence for the S-form splice variant of the At1g25290 protein (SEQ ID NO:7).

FIG. 1H shows the cDNA sequence for a human UBAC2 splice variant, isoform 1 (SEQ ID NO:8).

FIG. 2A shows the amino acid sequence of the full or L-form At1g74130 protein (SEQ ID NO:9).

FIG. 2B shows the amino acid sequence of the 7/8 M-form At1g74130 protein (SEQ ID NO: 10).

FIG. 2C shows the amino acid sequence of the 6/7 S-form At1g74130 protein (SEQ ID NO:11).

FIG. 2D shows the amino acid sequence of the L-form At1g25290 protein (SEQ ID NO:12).

FIG. 2E shows the amino acid sequence of the S-form At1g25290 protein (SEQ ID NO:13).

FIG. 2F shows the amino acid sequence of isoform 1 of the human UBAC2 protein (SEQ ID NO:14).

FIG. 3 is a diagram of a bacterial expression vector for the L-form splice variant of At1g74130.

FIG. 4 is a diagram of a bacterial expression vector for the M-form splice variant of At1g74130, showing the 11 nucleotide insertion of this variant. The full sequence is shown in SEQ ID NO:3.

FIG. 5 is a diagram of a bacterial expression vector for the S-form splice variant of At1g74130, showing the 5 nucleotide insertion of this variant. The full sequence is shown in SEQ ID NO:4.

FIG. 6 is a diagram of a bacterial expression vector for the L-form splice variant of At1g25290, showing the 9 nucleotide RVL coding region of this variant. The full sequence is shown in SEQ ID NO:6.

FIG. 7 is a diagram of a bacterial expression vector for the S-form splice variant of At1g25290, showing the 9 nucleotide deletion (RVL coding region) of this variant. The full sequence is shown in SEQ ID NO:7.

FIG. 8 is a diagram of a bacterial expression vector for the human UBAC2 splice variant (isoform 1).

FIG. 9 is a diagram of a yeast expression vector for the L-form splice variant of At1g74130.

FIG. 10 is a diagram of a yeast expression vector for the M-form splice variant of At1g74130, showing the 11 nucleotide insertion of this variant. The full sequence is shown in SEQ ID NO:3.

FIG. 11 is a diagram of a yeast expression vector for the S-form splice variant of At1g74130, showing the 5 nucleotide insertion of this variant. The full sequence is shown in SEQ ID NO:4.

FIG. 12 shows results of experimental treatment of bacteria cells with L, M, and S splice variant proteins of At1g74130 and ampicillin.

FIG. 13 shows results of experimental treatment of bacteria cells with L, M, and S splice variant proteins of At1g74130 and tetracycline.

FIG. 14 shows results of experimental treatment of bacteria cells with L and S splice variants of At1g25290 rhomboid protein and ampicillin.

FIG. 15 shows results of experimental treatment of bacteria cells with human UBAC2 variant (isoform 1) and ampicillin.

FIG. 16 shows results of experimental treatment of bacteria cells with human UBAC2 variant (isoform 1) and tetracycline.

FIG. 17 shows experimental results of yeast sensitivity to the fungicides amphotericin B (AmB) and nystatin (Nys).

FIG. 18 shows experimental results of treatment of yeast with L, M, and S splice variants of At1g74130 rhomboid proteins alone and together with AmB and Nys.

FIG. 19 shows experimental results of treatment of yeast with the L and S splice variant proteins of At1g25290, AmB, and Nys.

FIG. 20 shows experimental results of treatment of yeast with human splice variant proteins of UBAC2, AmB, and Nys.

FIGS. 21A, 21B, and 21C show MTT assay results for MDA-MB-231 breast cancer cells after 24, 48, and 72 hours, respectively, of treatment.

FIGS. 22A, 22B, and 22C show MTT assay results for MDA-MB-468 breast cancer cells after 24, 48, and 72 hours, respectively, of treatment.

### DETAILED DESCRIPTION OF EMBODIMENTS

Two inactive plastid rhomboids of Arabidopsis are At1g74130 and At1g74140. Both genes have yet to be found in other plants with sequenced genomes. Although the At1g74130 gene sequence and the encoded rhomboid protein have been identified (GenBank™/EMBL Data Bank, Accession No. NM_202412), roles have yet to be determined for the protein and related products. At1g74130 has been shown to exhibit a limited ability to rescue the yeast rbd1Δ mutant in glycerol-supplemented media and to partially restore the pattern of Tic40 bands in yeast mitochondria. Tic40 is a plastid translocon component and is a recognizable substrate of the yeast mitochondrial Rbd1. The limited nature of At1g74130's ability to rescue rbd1Δ may partly reflect its inactive status. Although considered inactive, At1g74130 still appears to play roles in plant tissue development (discussed above). At1g74130 exists as three functional splice variants.

Another rhomboid found in the plastids of Arabidopsis is At1g25290. At1g25290 is categorized as an active rhomboid protease (Lemberg. M. K., et al., Functional and evolutionary implications of enhanced genomic analysis of rhomboid intramembrane proteases. Genome Res. 17: 1634-1646, 2007). At1g25290 exists as variant forms L (NM_102339.3) and S (NM-0011981641.1) (Sedivy-Haley, K., et al., Characterization of two alternative splice variants associated with the Arabidopsis rhomboid protein gene At1g25290. Botany 90:1252-1262, 2012). The difference between the L and S variants is the presence or absence of a short RVL putative cyclin-binding domain. This RVL domain is linked to cell cycle control and cell growth, such as in cancer cells. Variant L contains the RVL cyclin-binding domain, whereas variant S lacks these three amino acid residues.

The human UBAC2 variant is a fusion between a rhomboid protein sequence (considered a rhomboid pseudoprotease) and ubiquitin-associating domains (gene ID 337827; XM_006719947.1; NM_001144072.1) (Christianson, J. C., et al., Defining human ERAD networks through an integrative mapping strategy. Nature Cell. Biol. 14: 93-105, 2012; Sawalha, A. H, et al., A putative functional variant within the UBAC2 gene is associated with increased risk of Behçet's disease. Arthritis and Rheumatism 63: 3607-3612, 2011).

However, embodiments described herein are not limited to these proteins and variants, as rhomboid protein is highly conserved across many species. Accordingly, active and inactive rhomboid proteins, polypeptides, and peptides corresponding to or derived from the examples given in Tables 1 and 2 may also be useful in the methods and compositions described herein.

As used herein, the term “inactive” means no known enzymatic activity typical of an enzyme or proteolytic enzyme.

Exemplary compositions, methods, and uses based on At1g74130, At1g25290, and UBAC2 and splice variants and functional equivalents thereof are described herein. In particular, functionally significant biological roles for three splice variants of At1g74130, two splice variants of At1g25290, and a splice variant of UBAC2 are described; however, the compositions, methods, and uses are not limited thereto. Such compositions and methods may be used for managing or modifying cellular processes in a wide range of organisms and cells. For example, these may include processes underlying antibiotic or drug resistance, processes underlying infections such as parasitic and fungal infections, processes for secretion and biofilm production, cancerous cell growth, and progression of diseases such as Alzheimer's, Parkinson's, and Type-2 Diabetes. Application of the compositions and methods may be more effective when used in combination with medical treatments, by, for example, enhancing or sustaining efficacy. The compositions and methods may also be used in combination with current and future pharmaceuticals as well as past pharmaceuticals that may have been discontinued as a result of low efficacy. For plants, the compositions and methods may also be used in combination with current and future agrochemicals as well as past agrochemicals that may have been discontinued as a result of low efficacy.

TABLE 1
List of rhomboid genes and proteins.

| | Preferred | | |
|---|---|---|---|
| Species | Name | Gene ID | Alternative Name(s) |
| Human | PARL | 55486 | |
| | RHBDF1 | 64285 | Rhomboid 5 homolog 1; iRhom1 |
| | RHBDF2 | 79651 | Rhomboid 5 homolog 2; |
| | | | iRhom2; veinlet-like 6 |
| | RHBDL1 | 9028 | Rhomboid 1; veinlet-like 1; |
| | | | RHBDL; RRP |
| | RHBDL2 | 54933 | Rhomboid 2; verinlet-like 2; |
| | | | RRP2 |
| | RHBDL3 | 162494 | Rhomboid 3; veinlet-like 3; |
| | | | RHBDL4; VRHO; ventrhoid; |
| | | | RRP3 |
| | RHBDD1 | 84236 | Rhomboid domain containing 1; |
| | | | RHBDL4 |
| | RHBDD2 | 57414 | Rhomboid domain containing 2; |
| | | | veinlet-like 7; RHBDL7 |
| | RHBDD3 | 25807 | Rhomboid domain containing 3; |
| | | | PTAG |
| | DERL1 | 79139 | Derlin-1; DER-1; DER1 |
| | DERL2 | 51009 | Derlin-2 |
| | DERL3 | 91319 | Derlin-3 |
| | UBAC2 | 337867 | UBA domain containing 2 |
| Mouse | Parl | 381038 | |
| | Rhbdf1 | 13650 | Rhomboid family 1; iRhom1 |
| | Rhbdf2 | 217344 | Rhomboid 5 homolog 2: iRhom2 |
| | Rhbdl1 | 117025 | Rhomboid; veinlet-like 1; |
| | Rhbdl2 | 230726 | Rhomboid 2; veinlet-like 2; |
| | | | RRP2 |
| | Rhbdl3 | 246104 | Rhomboid 3; veinlet-like 3; |
| | | | Rhbdl4; Rhomr1; Vrho |
| | Rhbdd1 | 76867 | Rhomboid domain containing 1; |
| | | | RHBDL4; RRP4 |
| | Rhbdd2 | 215160 | Rhomboid domain containing 2; |
| | | | RHBDL7; Usmg1 |
| | Rhbdd3 | 279766 | Rhomboid domain containing 3 |
| | Derl1 | 67819 | Derlin-1 |
| | Derl2 | 116891 | Derlin-2 |
| | Derl3 | 70377 | Derlin-3 |
| | Ubac2 | 68889 | Ubiquitin associated domain |
| | | | containing 2 |
| Arabidopsis | RBL1 | 817453 | At2g29050 |
| | RBL2 | 842616 | At1g63120 |
| | RBL3 | 830616 | At5g07250 |
| | RBL4 | 824545 | At3g53780 |
| | RBL5 | 841690 | At1g52580 |
| | RBL6 | 837831 | At1g12750 |
| | RBL7 | 828406 | At4g23070 |
| | RBL8 | 839113 | At1g25290; RBL10 |
| | RBL9 | 832642 | At5g25752; RBL11 |
| | RBL10 | 821028 | At3g17611; RBL14 |
| | RBL11 | 825015 | At3g58460; RBL15 |
| | RBL12 | 825121 | At3g59520; RBL13 |
| | At1g18600 | 838441 | PARL; RBL12 |
| | At1g74130 | 843753 | |
| | At1g74140 | 843754 | |
| | At3g07950 | 819986 | |
| | At5g25640 | 832640 | |
| | At5g38510 | 833839 | RBL9 |
| | At1g77860 | 844122 | KOMPEITO; KOM; RBL8 |
| | DER1 | 829054 | At4g29330 |
| | DER2.1 | 828269 | At4g21810 |
| | DER2.2 | 825823 | At4g04860 |
| Drosophila | rho | 38168 | Rhomboid-1; veinlet; RHO1 |
| | stet | 38169 | Rho-2 |
| | ru | 44856 | Roughoid; rho-3; ru-PA |
| | rho-4 | 32109 | |
| | rho-5 | 2768944 | iRhom |
| | rho-6 | 34640 | |
| | rho-7 | 36281 | PARL |
| | DER-1 | 33369 | |
| | DER-2 | 42005 | |
| C. elegans | ROM-1 | 184989 | |
| | ROM-2 | 183564 | |
| | ROM-3 | 191000 | |
| | ROM-4 | 8565076 | |
| | ROM-5 | 190260 | |
| S. cerevisiae | RBD1 | 852993 | Pcp1; YGR101W |
| | RBD2 | 855830 | YPL246C |
| | DER1 | 852500 | Der1p; YBR201W |

TABLE 2
List of alternative splice sequences sorted by organism.

| Human | | Mouse | | Arabidopsis | |
|---|---|---|---|---|---|
| PARL | NM_018622.5 | Parl | NM_001005767.4 | RBL1 At2g29050 | NM_128462.3 |
| | NM_001037639.1 | | XM_006522312.1 | | NM_001084504.1 |
| | XM_005247582.1 | Rhbdf1 | NM_010117.2 | RBL2 At1g63120 | NM_104990.3 |
| | XM_005247583.1 | | XM_006514492.1 | RBL3 At5g07250 | NM_120807.3 |
| | XM_005247584.1 | | XM_006514493.1 | | NM_001125710.1 |
| | XM_005247585.1 | | XM_006514494.1 | RBL4 At3g53780 | NM_115238.2 |
| | XM_005247586.1 | | XM_006514495.1 | | NM_180367.1 |
| | XM_005247587.1 | | XM_006514496.1 | RBL5 At1g52580 | NM_104136.2 |
| RHBDF1 | NM_022450.3 | | XM_006514497.1 | RBL6 At1g12750 | NM_101145.2 |
| | XM_005255494.1 | | XM_006514498.1 | | NM_001084059.2 |
| | XM_005255498.2 | | XM_006514499.1 | | NM_001198046.1 |
| | XM_006720918.1 | | XM_006514500.1 | RBL7 At4g23070 | NM_118436.1 |
| | XM_006720919.1 | | XM_006514501.1 | RBL8 At1g25290 | NM_102339.3 |
| | XM_006720920.1 | | XM_006514502.1 | | NM_001198164.1 |
| | XM_006720921 | | XM_006514503.1 | | RVL alternate spliced |
| | XM_006720922.1 | Rhbdf2 | NM_172572.3 | RBL9 At5g25752 | NM_147916.2 |
| RHBDF2 | NM_024599.5 | | NM_001167680.1 | RBL10 At3g17611 | NM_180275.2 |
| | NM_001005498.3 | | XM_006533108.1 | | NM_202600.1 |
| | XM_005257669.1 | Rhbdl1 | NM_144816.1 | | NM_001084701.1 |
| | XM_005257670.1 | | XM_006524021.1 | RBL11 At3g58460 | NM_115708.3 |
| | XM_005257672.2 | | XM_006524022.1 | | NM_001203197.1 |
| | XM_006722079.1 | Rhbdl2 | NM_183163.2 | RBL12 At3g59520 | NM_115814.2 |
| | XM_006722080.1 | | XM_006503025.1 | PARL At1g18600 | NM_101718.4 |
| | XM_006722081.1 | | XM_006503026.1 | At1g74130 | NM_106073.5 |
| | XM_006722082.1 | Rhbdl3 | NM_139228.3 | | NM_202413.1 |
| RHBDL1 | NM_001278720.1 | | XM_006533326.1 | | At1g74130(S) - 6/7 |
| | NM_001278721.1 | | XM_006533327.1 | | At1g74130(M) - 7/8 |
| | XM_005255665.1 | | XM_006533328.1 | At1g74140 | NM_106074.2 |
| | XM_005255666.2 | | XM_006533329.1 | | NM_001124127.1 |
| | XM_006720974.1 | | XM_006533330.1 | | NM_001084350.2 |
| | XM_006720975.1 | | XM_006533331.1 | | NM_001036203.1 |
| RHBDL2 | NM_017821.3 | Rhbdd1 | NM_029777.3 | | NM_001084351.2 |
| RHBDL3 | NM_138328.2 | | NM_001122685.1 | At3g07950 | NM_111674.2 |
| | XM_006721733.1 | | XM_006496574.1 | At5g25640 | NM_122475.1 |
| | XM_006721734.1 | Rhbdd2 | NM_146002.2 | At5g38510 | NM_123212.2 |
| | XM_006721733.1 | | XM_006504416.1 | | NM_001085212.1 |
| | XM_006721736.1 | | XM_006504417.1 | At1g77860 | NM_106435.2 |
| RHBDD1 | NM_032276.3 | Rhbdd3 | NM_177370.3 | DER1 At4g29330 | NM_119078.4 |
| | NM_001167608.1 | | XM_006514713.1 | DER2.1 At4g21810 | NM_118301.6 |
| | XM_005246898.1 | | XM_006514714.1 | DER2.2 At4g04860 | NM_11672.3 |
| | XM_005246899.1 | | XM_006514715.1 | | |
| | XM_005246900.1 | | XM_006514716.1 | | |
| RHBDD2 | NM_001040456.1 | | XM_006514717.1 | | |
| | NM_001040457.1 | Derl-1 | NM_024207.4 | | |
| | XM_005250511.2 | Derl-2 | NM_033562.4 | | |
| RHBDD3 | NM_012265.1 | | NM_001291146.1 | | |
| | XM_005261504.2 | | NM_001291147.1 | | |
| | XM_006724224.1 | | NM_001291148.1 | | |
| DERL-1 | NM_024295.5 | Derl-3 | NM_024440.2 | | |
| | NM_001134671.2 | | XM_006514079.1 | | |
| | XM_006716657.1 | Ubac2 | NM_026861.2 | | |
| DERL-2 | NM_016041.3 | | XM_006519492.1 | | |
| | XM_005256672.2 | | XM_006519493.1 | | |
| | XM_006721539.1 | | | | |
| DERL-3 | NM_00113575.1 | | | | |
| | NM_001002862.2 | | | | |
| | NM_198440.3 | | | | |
| | XM_006725410.1 | | | | |
| | XM_006725411.1 | | | | |
| | XM_005261824.2 | | | | |
| | XM_006724371.1 | | | | |
| UBAC2 | NM_001144072.1 | | | | |
| | NM_177967.3 | | | | |
| | XM_006719947.1 | | | | |
| | XM_006719948.1 | | | | |
| | XM_006719949.1 | | | | |

| Drosophila | | C. elegans | | S. cerevisiae | |
|---|---|---|---|---|---|
| rho | NM_079159.3 | ROM-1 | NM_065628.2 | PCP1 | NM_001181230.1 |
| (rhomboid) | NM_001274326.1 | ROM-2 | NM_066719.2 | RBD2 | NM_001184060.1 |
| stet | NM_176273.2 | ROM-3 | NM_070612.2 | DER1 | NM_001178549.1 |
| | NM_176272.2 | ROM-4 | NM_001083250.4 | | |
| ru (roughoid) | NM_080051.2 | | NM_001047548.3 | | |
| rho-4 | NM_167290.2 | | NM_001047549.2 | | |
| rho-5 | NM_205957.3 | ROM-5 | NM_058724.5 | | |
| rho-6 | NM_176024.2 | | | | |
| rho-7 | NM_078980.3 | | | | |
| DER-1 | NM_134788.4 | | | | |
| | NM_001272963.1 | | | | |
| DER-2 | NM_142296.3 | | | | |

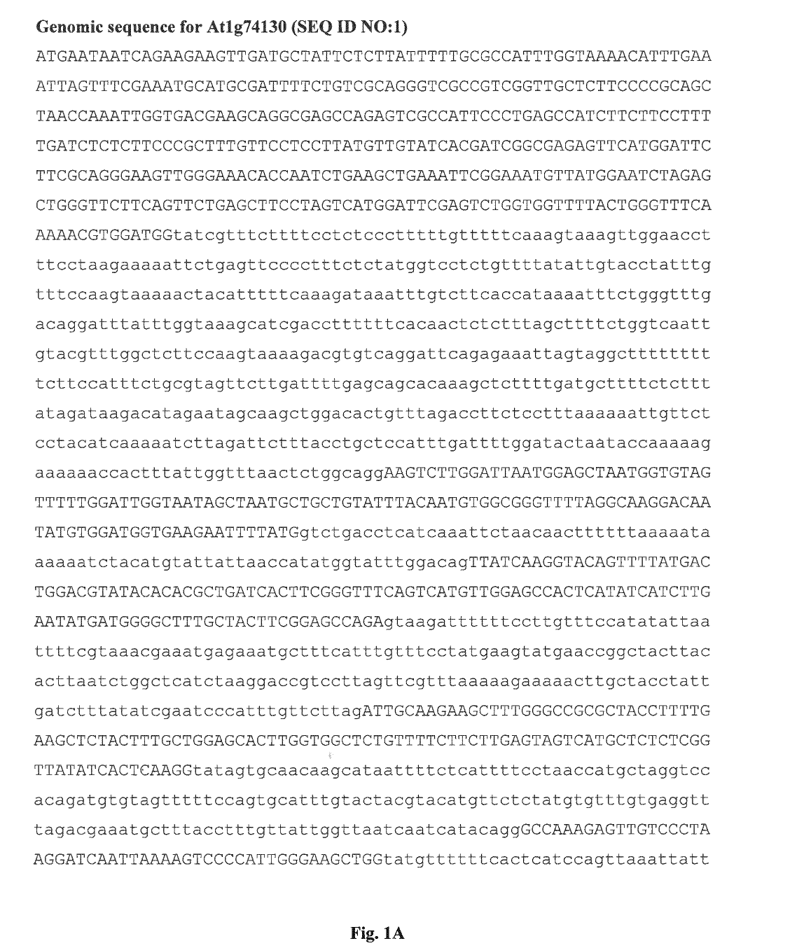

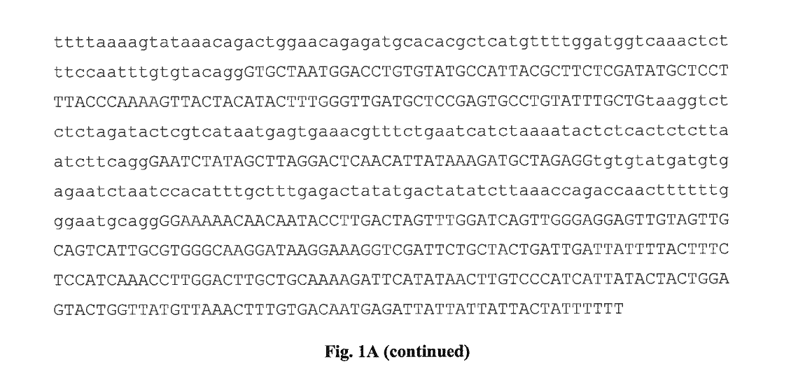

Some embodiments provide methods of using isolated and/or recombinant nucleic acids (DNA or RNA) that are characterized by (1) their ability to hybridize to (a) a nucleic acid encoding a protein or polypeptide, such as a nucleic acid having the sequence of SEQ ID NO:1 (FIG. 1A) or (b) a portion thereof (e.g., a portion comprising the minimum nucleotides required to encode a functional At1g74130 protein, such as SEQ ID NO:2 (FIG. 1B), SEQ ID NO:3 (FIG. 1C), or SEQ ID NO:4 (FIG. 1D); or their ability to hybridize to (c) a nucleic acid encoding a protein or polypeptide, such as a nucleic acid having the sequence of SEQ ID NO: 5 (FIG. 1E) or (d) a portion thereof (e.g., a portion comprising the minimum nucleotides required to encode a functional At1g25290 protein, such as SEQ ID NO:6 (FIG. 1F), SEQ ID NO:7 (FIG. 1G); or their ability to hybridize to (e) a nucleic acid encoding a protein or polypeptide, such as a nucleic acid having the sequence of SEQ ID NO:8 (FIG. 1H) or a portion thereof (e.g., a portion comprising the minimum nucleotides required to encode a functional UBAC2 protein; or by (2) their ability to encode a polypeptide having the amino acid sequence of At1g74130, e.g., SEQ ID NO:9 (FIG. 2A), or to encode functional equivalents thereof, such as SEQ ID NO:10 (FIG. 2B) or SEQ ID NO:11 (FIG. 2C), or their ability to encode a functional equivalent of a polypeptide having the amino acid sequence of At1g25290, such as SEQ ID NO:12 (FIG. 2D) or SEQ ID NO:13 (FIG. 2E), or their ability to encode a functional equivalent of a polypeptide having the amino acid sequence of UBAC2, such as SEQ ID NO:14 (FIG. 2F); e.g., a polypeptide which when incorporated into a particular membrane (such as an envelope membrane of a plastid or a mitochondrion) alters or impacts the transport, integration, cleavage, processing or functionality of like molecules in the same manner as At1g74130, At1g25290, or UBAC2; or by (3) both of (1) and (2). A functional equivalent of At1g74130, At1g25290, or UBAC2, therefore, would have a similar amino acid sequence and similar characteristics to, or perform in substantially the same way as the At1g74130, At1g25290, or UBAC2 protein and variants and equivalents thereof. A nucleic acid which hybridizes or aligns to a nucleic acid encoding the At1g74130, At1g25290, or UBAC2 polypeptide such as SEQ ID NO:1, SEQ ID NO:5, or SEQ ID NO:8 can be double- or single-stranded. Hybridization to DNA such as DNA having the sequence of SEQ ID NO:1, SEQ ID NO:5, or SEQ ID NO:8 includes hybridization or alignment to the strand shown or its complementary strand. The same practice applies to any of the mentioned rhomboids, such as genomic sequences for At1g25290, UBAC2, the rhomboids listed in Tables 1 and 2, and the rhomboids from species beyond those highlighted in Tables 1 and 2.

In one embodiment, the amino acid sequence of a functional equivalent of an At1g74130 polypeptide has at least 20% identity to SEQ ID NO:9, SEQ ID NO: 10, or SEQ ID NO: 11. For example, a functional equivalent may have at least one modification, such as a substitution, deletion, addition, point mutation, fusion, or hybrid, relative to SEQ ID NO:9, SEQ ID NO:10, or SEQ ID NO:11. In one embodiment, the amino acid sequence of a functional equivalent of an At1g25290 polypeptide has at least 20% sequence identity to SEQ ID NO:12 or SEQ ID NO:13. For example, a functional equivalent may have at least one modification, such as a substitution, deletion, or point mutation, relative to SEQ ID NO:12 or SEQ ID NO:13. In one embodiment, the amino acid sequence of a functional equivalent of an UBAC2 polypeptide has at least 20% identity to SEQ ID NO:14. For example, a functional equivalent may have at least one modification, such as a substitution, deletion, or point mutation, relative to SEQ ID NO: 14.

In various embodiments, the amino acid sequence identity of a functional equivalent of an At1g74130 polypeptide is at least about 30%, or 40%, or 50% to SEQ ID NO:9, SEQ ID NO:10, or SEQ ID NO:11. In various embodiments, the amino acid sequence identity of a functional equivalent of an At1g25290 polypeptide is at least about 30%, or 40%, or 50% to SEQ ID NO:12 or SEQ ID NO:13. In various embodiments, the amino acid sequence identity of a functional equivalent of an UBAC2 polypeptide is at least about 30%, or 40%, or 50% to SEQ ID NO:14.

In further embodiments, the amino acid sequence identity of a functional equivalent of an At1g74130 polypeptide is at about 80% or about 90% to SEQ ID NO:9, SEQ ID NO:10, or SEQ ID NO: 11. In further embodiments, the amino acid sequence identity of a functional equivalent of an At1g25290 polypeptide is at about 80% or about 90% to SEQ ID NO:12 or SEQ ID NO:13. In further embodiments, the amino acid sequence identity of a functional equivalent of an UBAC2 polypeptide is at about 80% or about 90% to SEQ ID NO:14.

Isolated and/or recombinant nucleic acids meeting these criteria comprise nucleic acids having sequences identical to sequences of naturally occurring At1g74130, At1g25290, or UBAC2 genes and portions thereof, or variants of the naturally occurring genes. Such variants include mutants differing by the addition, deletion, or substitution of one or more nucleotides, modified nucleic acids in which one or more nucleotides are modified (e.g., DNA or RNA analogs), and mutants comprising one or more modified nucleotides.

Such nucleic acids, including DNA or RNA, can be detected and isolated by hybridization under high stringency conditions or moderate stringency conditions, for example, which are chosen so as to not permit the hybridization of nucleic acids having non-complementary sequences. “Stringency conditions” for hybridizations is a term of art which refers to the conditions of temperature and buffer concentration which permit hybridization of a particular nucleic acid to another nucleic acid in which the first nucleic acid may be perfectly complementary to the second, or the first and second may share some degree of complementarity which is less than perfect. For example, certain high stringency conditions can be used which distinguish perfectly complementary nucleic acids from those of less complementarity. Methods for determining “high stringency conditions” and “moderate stringency conditions” for nucleic acid hybridizations are known in the art and are not described here. For example, reference is made to pages 2.10.1-2.10.16 (see particularly 2.10.8-11) and pages 6.3.1-6 in Current Protocols in Molecular Biology (Ausubel, F. M. et al., eds., Vol. 1, containing supplements up through Supplement 29, 1995), the teachings of which are hereby incorporated by reference. The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high or moderate stringency conditions can be determined empirically.

For example, high stringency hybridization procedures can (1) employ low ionic strength and high temperature for washing, such as 0.015M NaCl/0.0015M sodium citrate, pH 7.0 (0.1×SSC) with 0.1% sodium dodecyl sulfate (SDS) at 50° C.; (2) employ during hybridization 50% (vol/vol) formamide with 5×Denhardt's solution (0.1% weight/volume highly purified bovine serum albumin/0.1% wt/vol Ficoll/0.1% wt/vol polyvinylpyrrolidone), 50 mM sodium phosphate buffer at pH 6.5 and 5×SSC at 42° C.; or (3) employ hybridization with 50% formamide, 5×SSC, 50 mM sodium phosphate (pH 6.8), 0.1% sodium pyrophosphate, 5×Denhardt's solution, sonicated salmon sperm DNA (50 .mu.g/ml), 0.1% SDS, and 10% dextran sulfate at 42° C., with washes at 42° C. in 0.2×SSC and 0.1% SDS.

By varying hybridization conditions from a level of stringency at which no hybridization occurs to a level at which hybridization is first observed, conditions which will allow a given sequence to hybridize with the most similar sequences in the sample can be determined.

Exemplary conditions are described in Krause, M. H. and S. A. Aaronson (1991) Methods in Enzymology, 200:546-556. Also, see especially page 2.10.11 in Current Protocols in Molecular Biology (supra), which describes how to determine washing conditions for moderate or low stringency conditions. Washing is the step in which conditions are usually set so as to determine a minimum level of complementarity of the hybrids. Generally, from the lowest temperature at which only homologous hybridization occurs, a 1% mismatch between hybridizing nucleic acids results in a 1° C. decrease in the melting temperature Tₘ for any chosen SSC concentration. Generally, doubling the concentration of SSC results in an increase in Tₘ of about 17° C. Using these guidelines, the washing temperature can be determined empirically for moderate or low stringency, depending on the level of mismatch sought.

Isolated and/or recombinant nucleic acids that are characterized by their ability to hybridize or align to (a) a nucleic acid encoding a At1g74130 polypeptide, such as the nucleic acid depicted as SEQ ID NO: 1, a nucleic acid encoding a At1g25290 polypeptide, such as the nucleic acid depicted as SEQ ID NO:5, or a nucleic acid encoding a UBAC2 polypeptide, such as the nucleic acid depicted as SEQ ID NO:8, (b) the complement of SEQ ID NO:1, SEQ ID NO:5, or SEQ ID NO:8, or (c) a portion of (a) or (b) (e.g., under high or moderate stringency conditions), may further encode a protein or polypeptide having at least one functional characteristic of a At1g74130, At1g25290, or UBAC2 polypeptide or binding of antibodies that also bind to non-recombinant At1g74130, At1g25290, or UBAC2. The catalytic or binding function of a protein or polypeptide encoded by the hybridizing or aligning nucleic acid may be detected by standard enzymatic or interaction assays for activity or binding. Enzymatic assays, complementation tests, or other suitable methods can also be used in procedures for the identification and/or isolation of nucleic acids which encode a polypeptide such as a polypeptide of the amino acid sequence SEQ ID NO:9, SEQ ID NO:10, SEQ ID NO:11, SEQ ID NO:12, SEQ ID NO:13, or SEQ ID NO:14 or a functional equivalent thereof. The antigenic properties of proteins or polypeptides encoded by hybridizing or aligning nucleic acids can be determined by immunological methods employing antibodies that bind to a At1g74130 polypeptide such as immunoblot, immunoprecipitation and radioimmunoassay. PCR methodology, including RAGE (Rapid Amplification of Genomic DNA Ends), can also be used to screen for and detect the presence of nucleic acids which encode At1g74130, At1g25290, or UBAC2-like proteins and polypeptides, and to assist in cloning such nucleic acids from genomic DNA. PCR methods for these purposes are known in the art and will not be described here.

The nucleic acids are used for producing proteins or polypeptides which, in accordance with embodiments described herein, may be incorporated into cellular membranes whereupon they provide or facilitate functions such modifying the incorporation or functionality or processing of substances into or in the membrane. The “substances” may be, for example, proteinaceous molecules, such as proteins, peptides (including polypeptides), and molecules with peptide bonds, or nonpeptide compounds.

In one embodiment, DNA containing all or part of the coding sequence for a At1g74130, At1g25290, or UBAC2 polypeptide, or DNA which hybridizes to DNA having the sequence SEQ ID NO:1, SEQ ID NO:5, or SEQ ID NO:8, is incorporated into a vector for expression of the encoded polypeptide in suitable host cells. The encoded polypeptide consisting of At1g74130, At1g25290, or UBAC2 or a functional equivalent thereof is capable of modifying or impacting functionality of substances, such as those described above. The term “vector” as used herein refers to a nucleic acid molecule capable of transporting another nucleic acid to which it has been linked. A vector, therefore, includes a plasmid or viral DNA molecule into which another DNA molecule can be inserted without disruption of the ability of the molecule to replicate itself. The terms “translocating” or “translocation” mean the transport of substances across at least one cellular membrane from one part of the cell to another or into or out of the cell or organelle (i.e., import or secrete) or into a periplasmic space (i.e., export) such as that found in bacteria (e.g., E. coli) between the inner and outer membranes.

Nucleic acids referred to herein as “isolated” are nucleic acids separated away from the nucleic acids of the genomic DNA or cellular RNA of their source of origin (e.g., as it exists in cells or in a mixture of nucleic acids such as a library), and may have undergone further processing. Isolated nucleic acids include nucleic acids obtained by methods described herein, similar methods or other suitable methods, including essentially pure nucleic acids, nucleic acids produced by chemical synthesis, by combinations of biological and chemical methods, and recombinant nucleic acids which are isolated. Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes. Recombinant nucleic acids are also those that result from recombination events that occur through the natural mechanisms of cells, but are selected for after the introduction to the cells of nucleic acids designed to allow or make probable a desired recombination event. Portions of the isolated nucleic acids which code for polypeptides having a certain function can be identified and isolated by, for example, the method of Jasin, M., et al., U.S. Pat. No. 4,952,501.

Those of skill in the art will appreciate the numerous examples wherein the compositions and methods described herein result in commercial advantages in plants and other organisms. The following are several examples, not intended to be limiting, which describe the type of modifications of plants that are possible using directed, tissue-specific expression. Seed or pollen development or production can be impaired or inhibited. This is especially useful where there is a desire to prevent seeds of a valuable crop from being generated and harvested by competitors. The incorporation of truncated or mutant versions of At1g74130, At1g25290, or UBAC2 into organelle membranes could disturb function to the extent that an antisense-type of inhibition occurs in a particular tissue, for example, resulting in male sterility. Expression in pollen which disrupts or impairs its function could be especially useful to prevent escape of transgenic materials from crops. Transfer of inhibitory genetic material to wild populations of unwanted plants could produce herbicidal effects and valuable weed control. This type of population control can extend to aquatic organisms, such as the dinoflagellates which produce red tides, or blue-green algae which are responsible for toxic blooms in freshwater systems. Additionally, yields of crops harvested for vegetative materials could be increased by impairing the investment of energy in flower and seed production.

Other embodiments relate to compositions and methods using the proteins or polypeptides encoded by the nucleic acids. The proteins and polypeptides may be isolated and/or recombinant. Proteins or polypeptides referred to herein as “isolated” are proteins or polypeptides purified to a state beyond that in which they exist in cells. Isolated proteins or polypeptides include proteins or polypeptides obtained by methods described herein, similar methods or other suitable methods, and include essentially pure proteins or polypeptides, proteins or polypeptides produced by chemical synthesis or by combinations of biological and chemical methods, and recombinant proteins or polypeptides which are isolated. Proteins or polypeptides referred to herein as “substantially purified” have been isolated and purified, such as by one or more steps usually including column chromatography, differential precipitation, or the like, to a state which is at least about 10% pure. Proteins or polypeptides referred to herein as “recombinant” are proteins or polypeptides produced by the expression of recombinant nucleic acids.

Another embodiment relates to compositions and methods using the proteins or polypeptides encoded by the nucleic acids. The proteins and polypeptides isolated using various techniques described above, may be delivered to cells or membranes directly or indirectly with a suitable delivery vehicle. A delivery vehicle maybe based on natural delivery systems of cells or organisms or derived through synthetic means. Natural delivery systems may be based on antibodies, viruses, viral proteins, or other natural methods. Synthetic delivery means may be based on chemical or biochemical means, including fusion proteins or polypeptides, monoclonal antibodies, detergents such as deoxycholate and dodecyl D maltoside; lipids and fatty acids such as liposomes, micelles, lecithin, and ethyl oleate, solvents such as dimethyl sulfoxide, methanol, glycerol, glycol, alcohol, and any other chemical or biochemical that facilitates delivery of the proteins or polypeptides.

One embodiment relates to modifying (e.g., enhancing) protein or peptide transport across a cellular membrane by incorporating an isolated nucleic acid encoding a plastid-derived rhomboid protein, At1g74130 (SEQ ID NO:1) or SEQ ID NO:2, SEQ ID NO:3, or SEQ ID NO:4, or a functional equivalent thereof, into a cell or organism and maintaining the cell or organism under conditions appropriate for expression of the At1g74130 protein or an equivalent.

Another embodiment relates to modifying functionality of a protein or peptide in a cellular membrane by providing a rhomboid protein having the amino acid sequence of SEQ ID NO:9, SEQ ID NO: 10, or SEQ ID NO: 11, or a functional equivalent thereof, to a cell or organism.

Another embodiment relates to modifying (e.g., enhancing) protein or peptide transport across a cellular membrane by incorporating an isolated nucleic acid encoding a plastid-derived rhomboid protein, At1g25290 (SEQ ID NO:5) or SEQ ID NO:6 or SEQ ID NO:7, or a functional equivalent thereof, into a cell or organism and maintaining the cell or organism under conditions appropriate for expression of the At1g25290 protein or an equivalent.

Another embodiment relates to modifying functionality of a protein or peptide in a cellular membrane by providing a rhomboid protein having the amino acid sequence of SEQ ID NO:12 or SEQ ID NO:13, or a functional equivalent thereof, to a cell or organism.

Another embodiment relates to modifying (e.g., enhancing) protein or peptide transport across a cellular membrane by incorporating an isolated nucleic acid encoding a human cell-derived rhomboid protein, UBAC2 (SEQ ID NO:8), or a functional equivalent thereof, into a cell or organism and maintaining the cell or organism under conditions appropriate for expression of the UBAC2 protein or an equivalent.

Another embodiment relates to modifying functionality of a protein or peptide in a cellular membrane by providing a rhomboid protein having the amino acid sequence of SEQ ID NO:14, or a functional equivalent thereof, to a cell or organism.

Such methods may be used to effect or modify functionality of substances in a cellular membrane. The term “cellular membrane” is intended to include one or both of the double membranes of chloroplasts, mitochondria, nuclei, and bacteria, and also plasma membranes, thylakoids, cristae, vesicular membranes, Golgi membranes, membranes which comprise the endoplasmic reticulum, and the like. The term “cellular membrane” is also intended to include artificially-made membranes or vesicles, into which proteins and/or peptides as described herein may be incorporated, and used, for example, to facilitate or enhance translocation of integrated compounds.

The cellular membrane may be in any type of cell, such as single-celled prokaryotic organisms; i.e., bacteria and cyanobacteria (blue-green algae), protists (single-celled eukaryotic organisms), fungal, plant, and animal, including mammalian and human, as well as cell lines derived from any of these eukaryotic organisms.

In addition to transport of molecules into and out of organelles and cells, the compositions and methods described herein may be used to facilitate or enhance the incorporation of proteins and other molecules into cellular membranes. Like many proteins that form part of a membrane structure and facilitate transfer of molecules through the membrane, the proteins and peptides described herein are also capable of impacting the integration of substances into membrane structure.

The present disclosure describes for the first time that At1g74130, At1g25290, or UBAC2 proteins and peptides are membranous components which can be used for modifying the transport of substances across cellular membranes, or modifying the incorporation of substances into cellular membranes. The substances may be proteins, molecules containing peptide bonds, or nonproteinaceous molecules.

An aspect of the embodiments is the surprising finding that the proteins and peptides will incorporate and function in substantially any membrane, including the inner and outer membranes of prokaryotes such as E. coli, and not just the Arabidopsis chloroplast envelope where it occurs naturally. Even more surprising is the discovery that the proteins and peptides alter the transport of substances out of the bacterial cell; whereas, in a plastid, transport of proteins occurs from the cytosol into the intraorganellar sites of the organelle. That At1g74130, At1g25290, or UBAC2 is effective by itself, without requiring additional introduction of other components, is both unusual and advantageous. Further, this is the first time a eukaryotic rhomboid gene encoding a membranous protein component has been incorporated into a prokaryote and the protein expressed with functional activity.

In one embodiment, therefore, depending on the setting, translocation or integration of molecules is affected by the introduction of At1g74130, At1g25290, or UBAC2. Expression of At1g74130, At1g25290, or UBAC2 in bacteria appears to modify the level of protein translocation by lowering the secretion levels. Without being bound by theory, it is suggested that the lower levels result from alterations to the protein translocation process or from the reduction of pathways for transport. Further, it is suggested that the effects are stable because the newly introduced protein is not recognized as being bacterial in origin, thereby reducing the possibility of down-regulation. This is the first time that alteration of bacterial protein translocation has been caused by a plant rhomboid protein or peptide, and that the activity of the rhomboid protein or peptide also results in changes to protein expression. Together, these two features result in a novel and significant impact on the ultimate levels of translocated proteins and the subsequent ability of bacteria to tolerate ampicillin. The same compositions and methods may also be used to alter transport into plastids and other organelles of plants at levels and/or in a manner which is not found in a naturally-occurring organism.

Expression of At1g74130 in S. cerevisiae

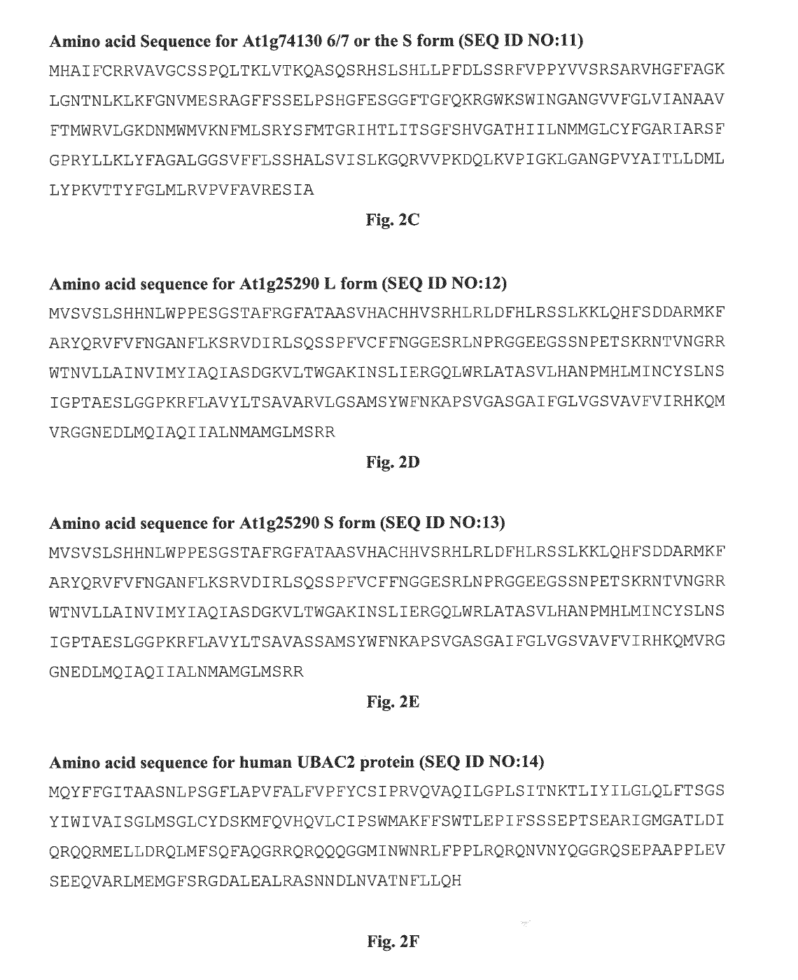

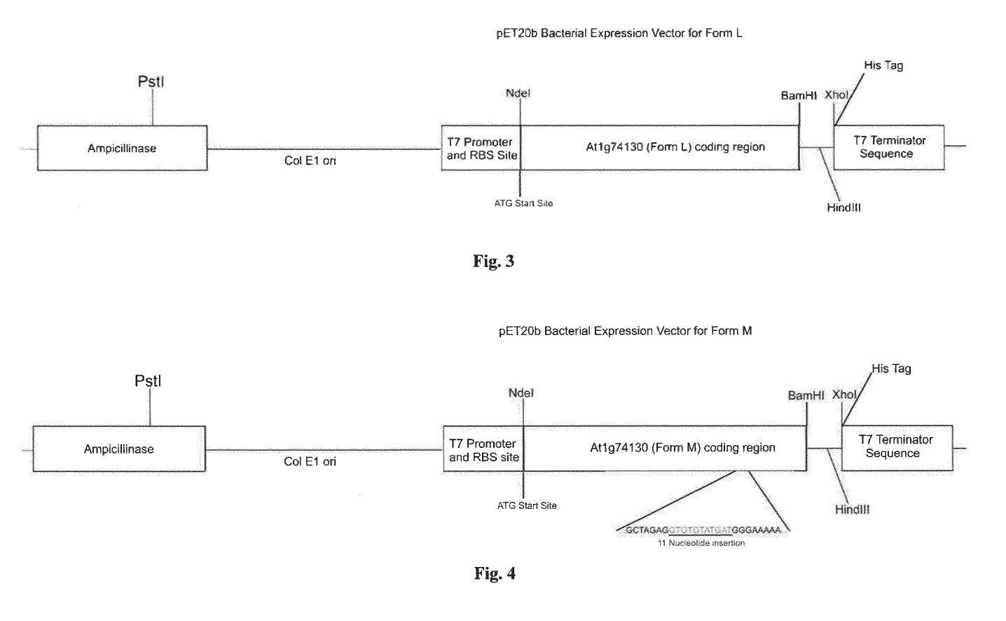

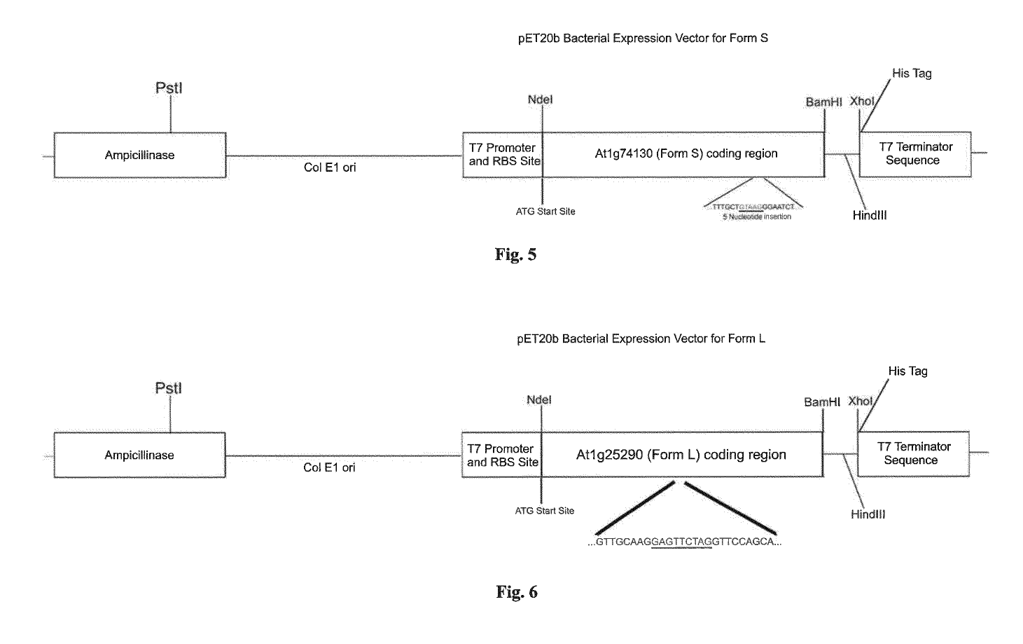

Introduction and expression of the At1g74130 rhomboid protein was tested in yeast cells and in particular, the yeast mitochondrial system. Introduction and expression of At1g74130 or a functional equivalent was accomplished by subcloning the corresponding cDNA sequence into vectors such as pACT, a bacteria-yeast shuttle cloning vector (see, e.g., FIGS. 9, 10, and 11). In the instance of pACT, gene expression was facilitated using either the constitutive promoter derived from the yeast alcohol dehydrogenase gene or the developmentally-regulated yeast promoter of the endogenous Rbd1 (PARL) mitochondrial rhomboid protease. Two alternative splice variants of At1g74130 were also tested and compared in the same manner. These two alternative splice variants were derived by RT-PCR cloning and tested along with the full version of At1g74130, which was also derived by RT-PCR cloning, as opposed to being obtained from resources available to users, such as the Arabidopsis Resources available at the Ohio State University and its consortium. The alternative splice variants designated At1g74130-6/7 and At1g74130-7/8 are shown in FIGS. 1C and 1D, respectively. The two variants tested were the same ones tested in bacteria (described below).

### Measurement of Effect on Yeast Mitochondrial Function, Cell Growth, and Susceptibility to the Fungicide Nystatin.

Interactions between At1g74130 and Mgm1-like proteins could slow down the fusion of mitochondria and their respiratory status, which would prevent efficient synthesis of ATP—a key respiratory product for cell function and growth. As seen in the above S. cerevisiae studies, yeast cells are generally smaller and appear to be impacted in the presence of At1g74130 or functional equivalents, such as the two alternative splice variants. Immunoblot analysis of the mitochondrial protein Mgm1 indicates that the cleavage of Mgm1 is impacted by the presence of At1g74130 (or its variants) from Arabidopsis. The ratio of cleaved Mgm1 versus uncleaved Mgm1 was impacted or altered. The change in this ratio appears to exert an impact on the respiratory status of the yeast cells. This impact was frequently observed as a higher percentage of small cells in the population, more frequent observations of small budding yeast cells, and higher levels of susceptibility to the antifungal agent nystatin. The lower level of antifungal resistance displayed by the At1g74130-expressing cells was reflected in the lower level of susceptibility, i.e., the diameter of the susceptibility circle was larger. The plasmid copy numbers were determined to be the same in the strains being compared so that copy number did not contribute to observed differences.

In addition to changes to levels of susceptibility, At1g74130-expressing cells, as a population, exhibited higher levels of small-sized cells when compared to the control cells. The smaller-sized cells may reflect an alteration to the growth progression of the yeast cells, which may also be a contributing factor in the corresponding lower level of antifungal susceptibility. Yeast cells were often observed budding as smaller-sized cells, a behaviour suggesting a slower or weaker progression through their life cycles. This slower pattern of growth is likely related to the impact exerted on their respiratory status.

Expression of Rhomboid Variants in E. coli

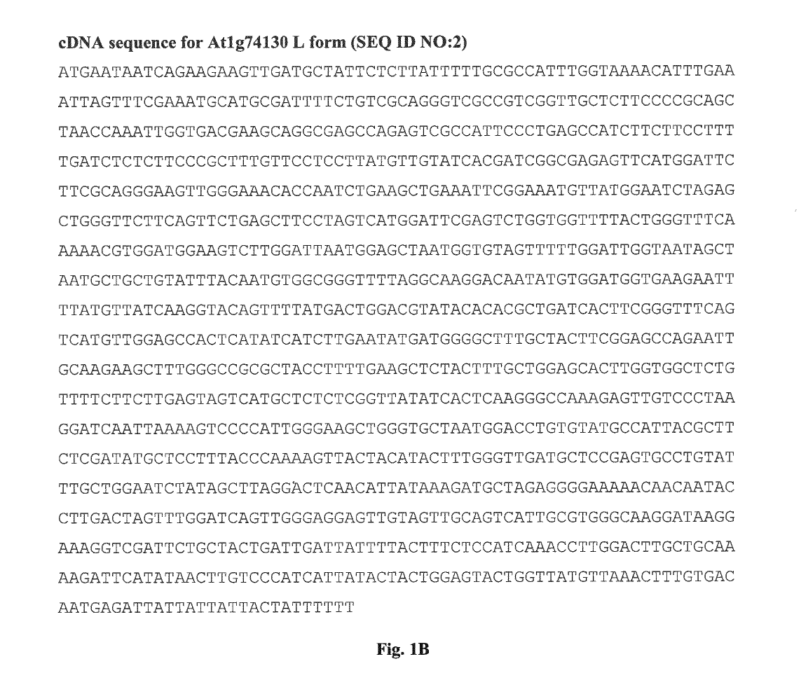

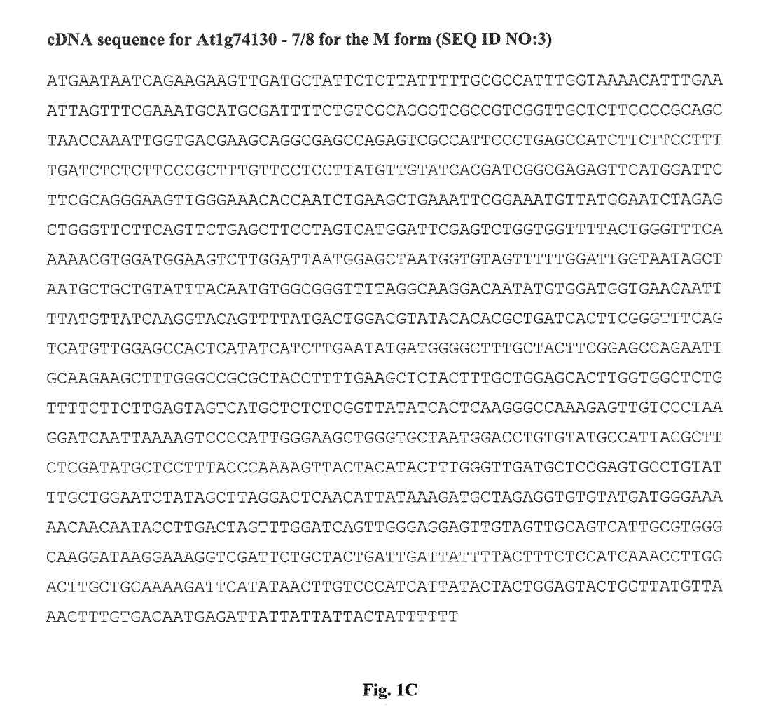

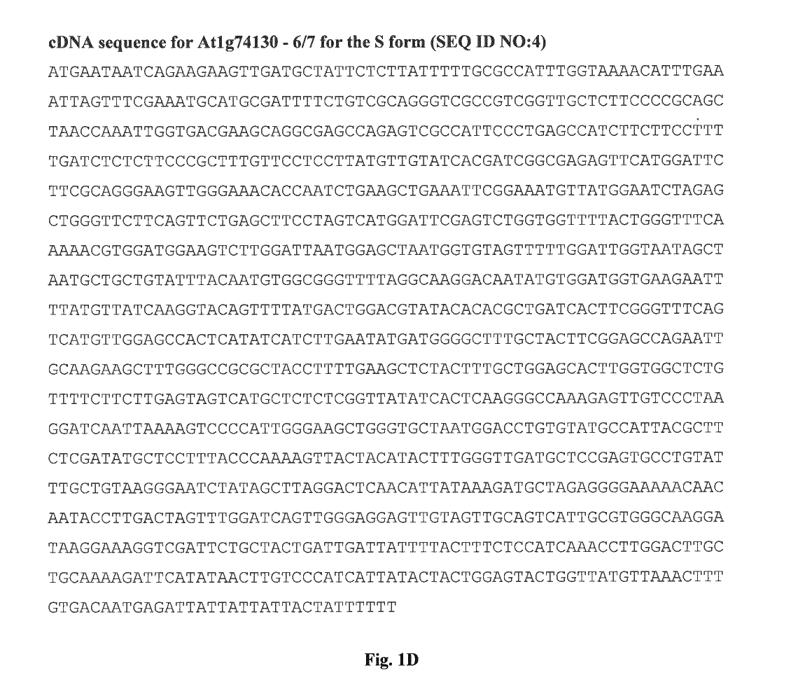

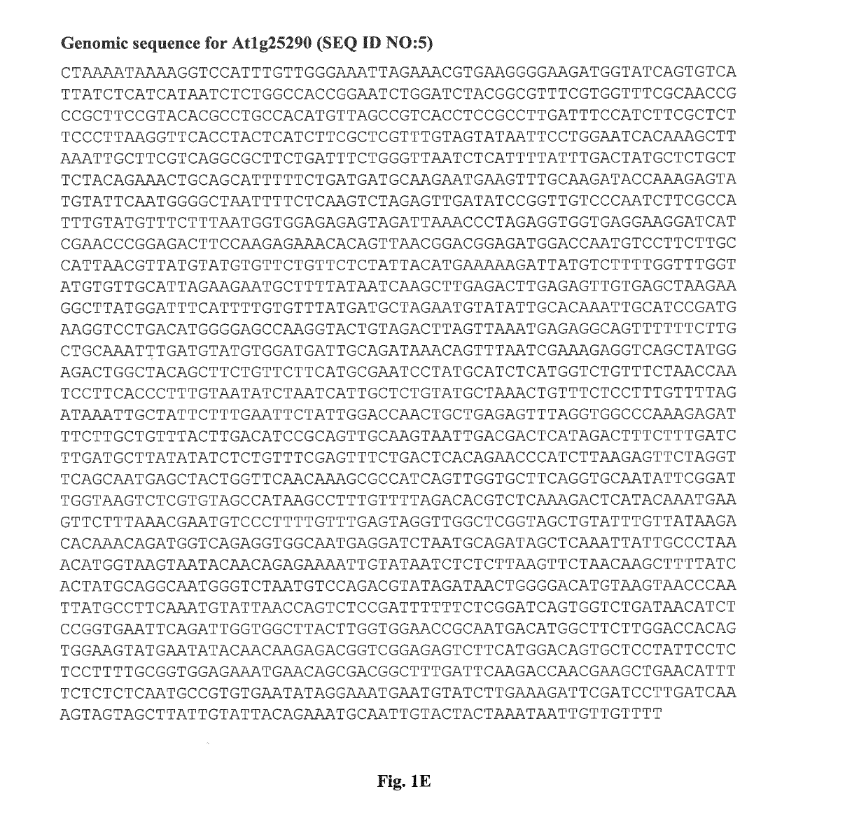

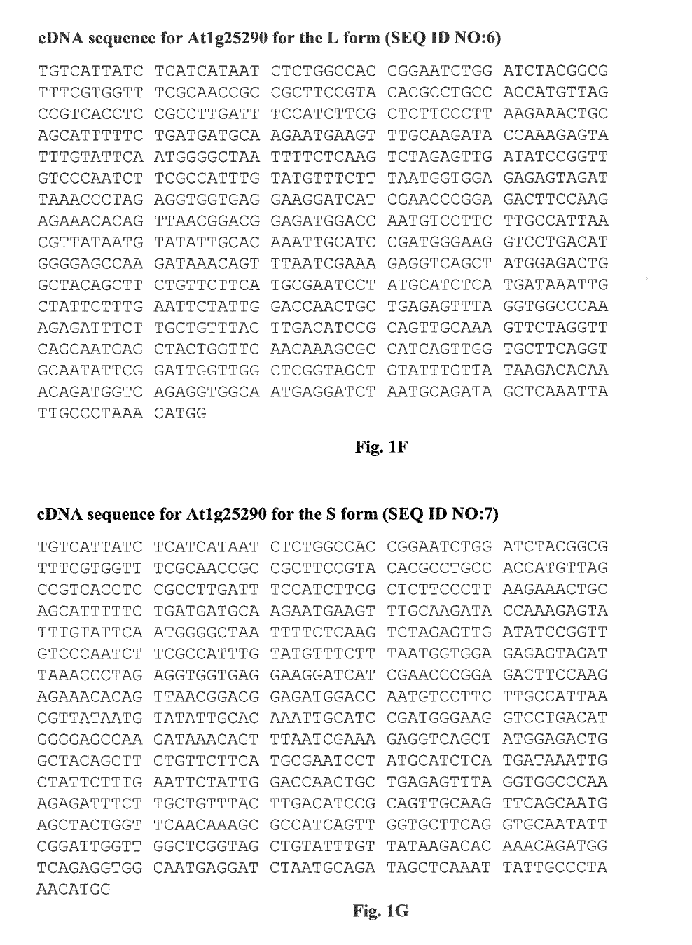

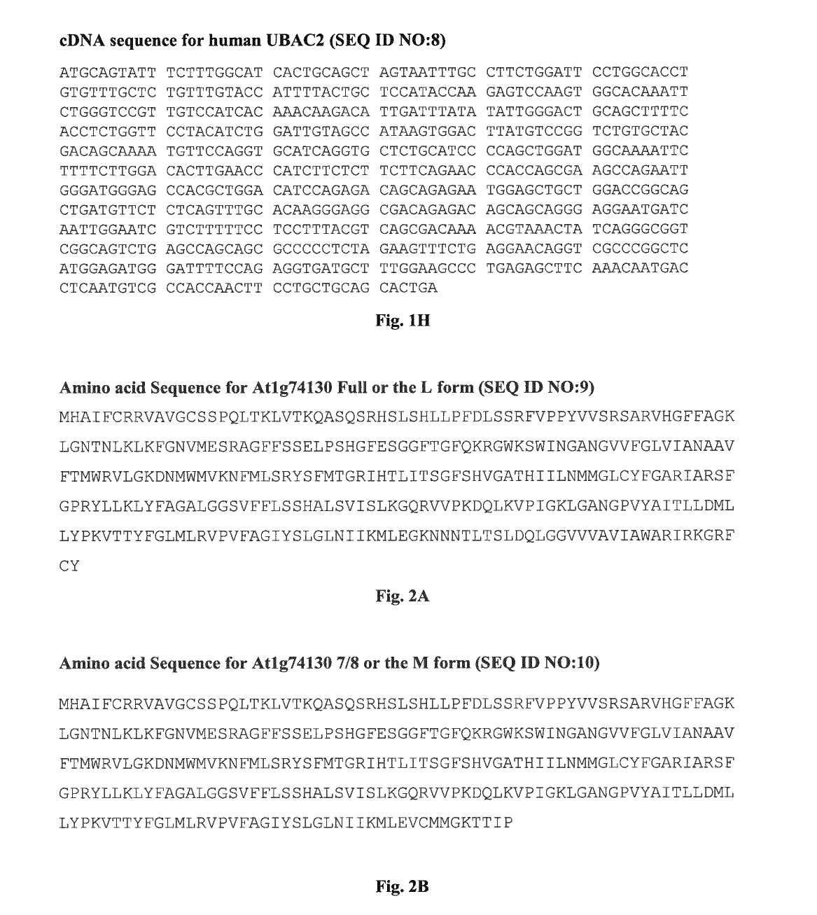

Introduction and expression of At1g74130 was tested in E. coli, by subcloning the corresponding cDNA sequence into a T7 promoter-containing plasmid vector (pET20b) and JM109(DE3), a bacterial strain commonly used for expressing foreign proteins. Two alternative splice variants of At1g74130 were derived by RT-PCR cloning and tested along with the full version of At1g74130, also derived by RT-PCR cloning (see, e.g., FIGS. 3, 4, and 5). The alternative splice variants designated 6/7 and 7/8 are shown in FIGS. 1C and 1D, respectively. A similar procedure was used to express the At1g25290 and UBAC2 variants (see FIGS. 6, 7, and 8).

### Measurement of β-Lactamase Protein Translocation and Synthesis Levels as an Antibiotic Susceptibility Marker

Alterations to protein translocation were investigated using the plasmid-borne multicopy gene, β-lactamase. The level of β-lactamase is a sensitive monitor of protein translocation and is translocated into the periplasm, where it detoxifies the antibiotic ampicillin, thereby conferring ampicillin-resistance to cells. Because the level of antibiotic resistance conferred also reflects the level of protein transport activity, the level of ampicillin resistance was determined for At1g74130 expressing cells and compared to that of control vector-containing cells on solid agar media containing increasing concentrations of ampicillin. The plasmid copy numbers were determined to be the same in both strains so that copy number did not contribute to differences in expression of β-lactamase. The At1g74130-expressing cells formed colonies with ampicillin concentrations at 1.25 mg/ml but begins to show significant susceptibility at higher concentration, whereas control cells (e.g., At1g74130 7/8) form colonies at 1.5 mg/ml and show susceptibility at higher concentrations of ampicillin.

The lower level of antibiotic resistance displayed by the At1g74130-expressing cells was reflected in the lower level of transported D-lactamase. Immunoblot analysis of cells grown in media containing ampicillin concentrations from 25 μg to 1.75 mg/ml showed that the level of processed β-lactamase is lower on a per cell basis in At1g74130-expressing cells than in control cells. These data demonstrate that At1g74130 affects the level of β-lactamase translocation and the overall level of β-lactamase synthesis, leading to a lower level of antibiotic resistance.

In addition to changes to levels of susceptibility, At1g74130-expressing cells, as a population, frequently exhibit higher levels of small colonies when compared to the colonies of control cells. The smaller size of the colonies at various stages of growth probably reflects an alteration to the growth progression of the bacterial cells, which may also be a contributing factor in the corresponding lower level of antibiotic susceptibility.

Further studies examined the effect of exogenous application of At1g25290 variants in bacteria and yeast, and show similar responses to those displayed by At1g74130. These are described below in the Examples.

Further studies examined the effect of exogenous application of At1g74130, At1g25290, and UBAC2 variants on sensitivity of cells to tetracycline. These are described below in the Examples.

Further studies examined the effect of exogenous application of At1g74130 on Pseudomonas aeruginosa growth and biofilm development. These are described below in the Examples.

Further studies examined the effect of exogenous application of At1g74130 variants on peptide delivery into Pseudomonas aeruginosa. These are described below in the Examples.

Further studies examined the effect of exogenous application of At1g74130 variants on the sensitivity of human breast cancer cells to a chemotherapeutic drug, lapatinib. These are described below in the Examples.

### Technological Applications

This disclosure provides, for the first time, evidence that a rhomboid protein can exert its impact without further manipulation. One surprising and important advantage of At1g74130 protein variants is their unique capacity to cause an effect alone, without requiring a multicomponent assembly which would be difficult to incorporate successfully into other organisms. As evidence of this advantage, data are provided herein demonstrating that At1g74130 variants impact the translocation of substances across bacterial (prokaryotic) membranes or yeast organelle membranes. In bacteria, the impact was manifested as enhanced susceptibility to the antibiotic ampicillin and slower colony growth. In Pseudomonas, At1g74130 variants impacted protein secretion and biofilm production or formation. In yeast, the impact was manifested as functional changes to the cell's mitochondrial system, cell growth, and enhanced susceptibility to the anti-fungal agent nystatin. In human breast cancer cells, At1g74130 variants increased the sensitivity of the cells to lower dose levels of the chemotherapy drug, lapatinib.

Therefore, for antibiotic-oriented applications, the described mechanism may work in at least two potential modes to alter the delivery or secretion or processing of factors underlying antibiotic resistance such as ampicillin. The two modes may work through one or more secretion mechanisms, such as the twin-arginine translocase complex and the SecYEG complex. Disturbance of the secretion process at any stage could impair the breakdown or disarming of antibiotics or the secretion of signals to activate anti-biotic resistance expression in other cells (quorum-sensing). There may be other less-characterized modes of impact that result in the same effect and these would be considered to fall within the scope of the embodiments described herein.

Similarly, At1g74130 could also be used in combination for the treatment of yeast infections, such as Candida infections. There are many species of pathogenic yeast, which when provided with the At1g74130 rhomboid protein may result in the hinderance of cell growth, cell development, and reduced survivability. Interactions between At1g74130 and Mgm1-like proteins could slow down the fusion of mitochondria into complex mitochondrial networks, and prevent efficient synthesis of ATP. As seen in the above S. cerevisiae studies, yeast cells are generally smaller and appear to be impacted in the presence of At1g74130. This phenomenon is enhanced when additional stress is applied to the yeast/infection.

Similarly, At1g74130 may also be used in combination for the treatment of bacterial infections, such as Pseudomonas infections. Pseudomonas is a gram-negative bacteria known to be pathogenic and a producer of biofilms.

The formation of biofilms is a complex process and requires the assembly of many components, such as carbohydrates, proteins and nucleic acids. The formation process also requires the expression and secretion of matrix proteins and enzymes. The secretion of such materials can occur through different types of secretory mechanisms, such as the Pseudopilin Type II secretion pathway in Pseudomonas. Any impact on the protein secretion mechanism may alter pathogenicity, growth, survival, biofilm production, and sensitivity to antimicrobial drugs.

The results reported herein demonstrate that specific variants of rhomboid proteins exhibit chemosensitizing capabilities in different settings. Specific variants, when added exogenously, can enhance the efficacies of antibacterial agents at non-lethal lower dose levels (the two drugs tested use different modes of action in E. coli) and two antifungals at non-lethal dose levels (the two drugs tested use different modes of action in yeast). Specific variants work or work better in different settings.

In the experiments reported here, specific rhomboid variants were effective against biofilm-producing Pseudomonas. The experiments here were conducted to assess the ability of rhomboid protein variants to impair the protein secretion process of Pseudomonas aeruginosa PAO1, a gram-negative bacterium known to be pathogenic and producers of biofilms. Any impact on the protein secretion mechanism may alter pathogenicity, growth and survival, biofilm production, and sensitivity to antimicrobial drugs.

In the experiments, Pseudomonas cells were required to express and secrete lipases in order to break down olive oil (the only external carbon source) and allow the acquisition of carbon from the media. The results suggested that the lipase secretion was impaired in cells treated with an At1g74130 rhomboid variant, such that they were not able to break down the olive oil, limiting their growth.

There are many species of Pseudomonas, which when provided with a rhomboid protein may result in the hinderance of cell growth, cell development, and biofilm development, and reduced survivability. In short, a rhomboid protein may be used as an antibiofilm agent.

Embodiments could similarly be used in the prevention or treatment of parasitic invasions, such as the parasites that cause malaria. For example, in some cases there are numerous microneme (MIC) proteins associated with the invasion of parasites. These MIC proteins function by interacting with rhomboid proteins or proteases. MIC proteins act as adhesin proteins that recognize human cells and are involved in the initiation of the invasion process. Rhomboid proteases within or on the parasite are activated or released after host cell invasion in order to cleave the MIC proteins from the eukaryotic cell, in order to evade host cell invasion. If the cleavage of these MIC proteins becomes impaired, it may be possible to prevent host cell invasion. The presence of externally-introduced At1g74130 or an equivalent thereof may prevent proper processing of MIC proteins, and thereby slow down the ability to invade host cells. This would provide sufficient time for the host cell immune system to detect parasites and launch an immune response by attacking the parasites.

Along the same strategy as above for MIC and malaria, embodiments be used in cancer immunotherapy approaches. For example, in some cases major histocompatibility complex (MHC) proteins are released from cell membranes by membrane-bound proteases called sheddases, or the like. The release of MHC proteins allows cancer cells to elude the immune system in human as in the case of malaria described above. The presence of externally-introduced At1g74130 or an equivalent thereof may prevent proper processing and shedding of MHC proteins, and thereby allowing the immune system to detect cancer cells more effectively and launch an immune response by attacking the cancer cells.

There may also be similar strategies for using At1g74130 or an equivalent thereof in the management of cancer cell growth, thereby improving the efficacy of medical treatments for this disease. The reported presence of a truncated transcript for human rhomboid proteins in breast cancer cells, termed RHBDD2, suggests that there may be a possibility of using one or more alternative splice variants of At1g74130 to manage the growth of cancer cells. As an example, such a possibility was reported for RHBDD1 by Han, J., et al., Lentivirus-mediated knockdown of rhomboid domain containing 1 inhibits colorectal cancer cell growth, Molecular Medicine Report. 12:377-381, 2015. For example, management could be achieved by introducing an At1g74130 alternative splice variant into the cell's environment through manipulations at the DNA, RNA, or protein levels. As most cancer cells are generated through mutations within developmental pathways, such as over-expression or increased secretion of growth hormones (homologous to Spitz), then the introduction of At1g74130 alternative splice variants into the cell's environment could interfere with the processing of such substrates or targets or prevent their release. Such impairment may slow down cell growth, thereby allowing medical treatments to reach higher levels of efficacy.

The same types of strategies could also be developed and utilized for the management of other diseases that involve rhomboid proteins or proteases. For example, over-cleavage of beta-amyloid has been suggested as one possible cause for the development of Alzheimer's disease. There is also evidence that rhomboid proteins play roles within the development of Parkinson's disease and diabetes.

### Applications in Sensitization

As used herein, the term “sensitization” generally refers to increasing sensitivity of a cell, tissue, or organ to a treatment or condition. In accordance with one aspect of embodiments disclosed herein, chemosensitization refers to using a chemical or compound that renders any pathogen, or diseased or dysfunctional cell more sensitive to another chemical or compound, drug, or treatment agent. Examples of applications of chemosensitization include, but are not limited to, mycotic infections, bacterial infections, cancerous cells, and viral infections. In accordance with another aspect of embodiments disclosed herein, sensitization refers to enhancing the desired effect of an agent on a cell, tissue, or organism. For example, one affect of a chemosensitizer agent may be to facilitate drug therapy at lower doses to reduce toxicity and side-effects.

According to one aspect of the embodiments described herein, novel compositions for overcoming drug resistance in disease, for sensitizing cells to treat disease, and methods for producing and using such compositions are provided.

In one embodiment the compositions and methods facilitate drug delivery to cells, tissues, and organisms. Administration of the compositions with a suitable pharmaceutical excipient as may be necessary can be carried out via any of the known and accepted modes of administration. Thus, administration can be, for example, intravenous, topical, subcutaneous, transcutaneous, intramuscular, oral, intra-joint, parenteral, intravaginal, peritoneal, intranasal, or by inhalation. Suitable sites of administration thus include, but are not limited to, skin, bronchial, gastrointestinal, anal, vaginal, eye, and ear.

As an example, the compositions can effect transfer of agents across the intestinal membrane into the blood stream. This is essential to enable oral drug delivery. Oral delivery of therapeutics remains the preferred route of administration due to increased patient compliance and a lower unit dose cost. To that end it is often desirable for an orally consumed pharmaceutical or nutraceutical bioactive material to be absorbed into the bloodstream through the wall of the small intestine or large intestine. The delivery vehicle which contains the bioactive agent must be able to pass intact through the stomach and must remain intact in the lumen of the intestine in order to be passed through the intestinal mucosa and deliver the bioactive agent into the blood stream. For example, enteric coatings are frequently used to encapsulate oral dosage forms to prevent damage to the active substance contained in the oral preparation by acids and enzymes in the stomach. Enteric coatings are used for example for preventing gastric enzymes from reacting with or destroying the active substance, preventing dilution of the active substance before it reaches the small intestine, ensuring that the active substance is not released until after the preparation has passed the stomach, and preventing damage to the bioactive agent because of the low pH in the stomach.

Another example is delivery of bioactive agents into the central nervous system. Currently, challenges exist to find effective drugs to treat neurological diseases. This is mainly due to the impermeability of the blood-brain barrier (BBB) that allows only 5% of more than 7000 small-molecule drugs available to treat only a tiny fraction of these diseases.

Examples of agents delivered to the central nervous system include, but are not limited to, agents useful for treating ischemia (e.g., using an anti-apoptotic drug), neurotransmitters and other agents for treating various conditions such as schizophrenia, Parkinson's disease, pain (e.g., morphine, the opiates), S-hydroxytryptamine receptor antagonist useful for treating conditions such as migraine headaches and anxiety. The compositions may be delivered within a variety of drug delivery vehicles not limited to emulsions, liposomes, and polymeric nanoparticles. Such delivery systems may also include methods of targeting to the blood brain barrier including receptor-specific ligands such as transferrin receptor (TR) ligands or antibodies or carriers demonstrated to cross the BBB such as glycopeptides (g7-NPs).

In another aspect, the compositions and methods can be utilized for topical administration and enhance drug delivery. For topical administration, the composition is administered in any suitable format, such as a lotion or a transdermal patch. The composition may be formulated as a solution, gel, ointment, cream, suspension, and the like, as are well known in the art. In some embodiments, administration is by means of a transdermal patch.

The compositions and methods may also be used to enhance administration of drugs through the respiratory tract. The respiratory tract, which includes the nasal mucosa, hypopharynx, and large and small airway structures, provides a large mucosal surface for drug absorption. The enhanced penetration of the conjugated agents into and across one or more layers of the epithelial tissue that is provided by the delivery-enhancing transporters of the invention results in amplification of the advantages that respiratory tract delivery has over other delivery methods. For example, lower doses of an agent are often needed to obtain a desired effect, a local therapeutic effect can occur more rapidly, and systemic therapeutic blood levels of the agent are obtained quickly. Rapid onset of pharmacological activity can result from respiratory tract administration. Moreover, respiratory tract administration generally has relatively few side effects.

The compositions and methods are also useful for transdermal delivery of anti-infective agents. For example, antibacterial, antifungal and antiviral agents can be conjugated to the delivery-enhancing transporters. Antibacterial agents are useful for treating conditions such as acne, cutaneous infections, and the like. Examples of antibiotics include clindamycin and erythromycin. Antifungal agents can be used to treat tinea corporis, tinea pedis, onychomycosis, candidiasis, tinea versicolor, and the like. Antifungal agents are also useful for treating onychomycosis. Examples of antifungal agents include, but are not limited to, azole antifungals such as itraconazole, myconazole and fluconazole. Examples of antiviral agents include, but are not limited to, acyclovir, famciclovir, and valacyclovir. Such agents are useful for treating viral diseases, e.g., herpes.

In other embodiments, the compositions and methods are useful for delivering cosmetics and agents that can treat skin conditions. Target cells in the skin that are of interest include, for example, fibroblasts, epithelial cells and immune cells. For example, the transporters provide the ability to deliver compounds such as anti-inflammatory agents to immune cells found in the dermis. Glucocorticoids (adrenocorticoid steroids) are among the compounds for which delivery across skin can be enhanced by the delivery-enhancing transporters of the invention. Conjugated glucocorticoids of the invention are useful for treating inflammatory skin diseases, for example. Exemplary glucocorticoids include, e.g., hydrocortisone, prenisone (deltasone) and predrisonlone (hydeltasol). Examples of particular conditions include eczema (including atopic dermatitis, contact dermatitis, allergic dermatitis), bullous disease, collagen vascular diseases, sarcoidosis, Sweet's disease, pyoderma gangrenosum, Type I reactive leprosy, capillary hemangiomas, lichen planus, exfoliative dermatitis, erythema nodosum, hormonal abnormalities (including acne and hirsutism), as well as toxic epidermal necrolysis, erythema multiforme, cutaneous T-cell lymphoma, discoid lupus erythematosus, and the like.

In further embodiments the compositions and methods can be used in combination with bioactive agents and drug compounds. The drug compound may be at least one drug compound selected from the group consisting of, but not limited to: amino acids, analgesic drugs, anti-inflammatory drugs, anthelmintics, antibacterials, aminoglycosides, beta lactam antibiotics, glycopeptides, penicillins, quinolones, sulphonamides, tranquilizers, cardiac glycosides, anti-parkinson agents, antidepressants, anti-neoplastic agents, immunosuppressants, antiviral agents, antibiotic agents, antifungal agents, antimicrobial agents, appetite suppressants, anti-emetics, antihistamines, antimigraine agents, coronary, cerebral or peripheral vasodilators; antianginals, calcium channel blockers, hormonal agents, contraceptive agents, antithrombotic agents, diuretics, antihypertensive agents, chemical dependency drugs, local anesthetics, corticosteroids, dermatological agents, vitamins, steroids, azole derivatives, nitro compounds, amine compounds, oxicam derivatives, mucopolysaccharides, opoid compounds, morphine-like drugs, fentany derivatives and analogues, prostaglandins, benzamides, peptides, xanthenes, catecholamines, dihydropyridines, thiazides, sydnonimines, polysaccharides, cholesterol lowering agents, phytochemicals, and antioxidants, or any derivative of the aforementioned. The drug classes mentioned above are listed for illustrative purposes.

In various embodiments, the compositions and methods include a vehicle such as a carrier and/or excipient (including but not limited to buffers, carbohydrates, mannitol, proteins, polypeptides or amino acids such as glycine, antioxidants, bacteriostats, chelating agents, suspending agents, thickening agents and/or preservatives), water, oils, saline solutions, aqueous dextrose and glycerol solutions, other pharmaceutically acceptable auxiliary substances as required to approximate physiological conditions, such as buffering agents, tonicity adjusting agents, wetting agents and the like. It will be recognized that, while any suitable carrier known to those of ordinary skill in the art may be employed to administer the compositions of this invention, the type of carrier will vary depending on the mode of administration. Compounds may also be encapsulated within liposomes using well-known technology. Biodegradable microspheres may also be employed as carriers for the compositions of this invention. Suitable biodegradable microspheres are shown, for example, in U.S. Pat. Nos. 4,897,268; 5,075,109; 5,928,647; 5,811,128; 5,820,883; 5,853,763; 5,814,344; and 5,942,252.

Formulations in accordance with these embodiments may take the form of solid, semi-solid, lyophilized powder, or liquid dosage forms, such as, for example, tablets, pills, capsules, powders, solutions, suspensions, emulsions, suppositories, retention enemas, creams, ointments, lotions, aerosols, eye drops, or the like, preferably in unit dosage forms suitable for simple administration of precise dosages. The compositions typically include a conventional pharmaceutical carrier or excipient and may additionally include other medicinal agents, carriers, adjuvants, and the like.

Embodiments are further described by way of the following non-limiting examples.

### Example 1

### Plant Material and the Propagation Regime Used

All plants were cultivated in growth cabinets with the following regime: 21° C., 70% humidity, 150-200 μmol·m⁻²·s⁻ lighting (fluorescent and incandescent lighting), and a 16:8 h light:dark photoperiod. The conditions used were according to the information posted on the Arabidopsis Biological Resource Center website at the time of this study (http://abrc.osu.edu). Sown seeds were stratified for 3 days prior to transferring to the growth chambers. The plants received daily watering and weekly fertilization. The At1g73140 mutant Arabidopsis line (SALK_142411c) and its parental control CS60000 (wild-type) were acquired from the Arabidopsis Biological Resource Center (Ohio State University). The parental CS60000 (wild-type) was used in the work described here to produce the expression plasmids for bacteria and yeast. The mutant line SALK_142411c was used for experimental analysis and comparisons as reported in Sedivy-Haley et al., 2012.

### Example 2

### Procedures for Analyzing At1g74130 Transcript Structure in Arabidopsis Plant Tissues

Total RNA were isolated from leaves of the parental line CS60000 Arabidopsis plants and processed with a DNase treatment step using Qiagen kits (RNeasy kit and the RNase-Free DNase set, Qiagen, Hilden, Germany). Analysis of At1g74130 transcript structure (accession number NM_00119864) was also conducted using Qiagen PCR kits (One-Step RT-PCR kit, Qiagen, Hilden, Germany).

Tissue samples, such as developing leaves, were collected during the light period between 11:00 and 13:00 h to accommodate circadian effects on transcription. When possible, RT-PCR assays were performed with different total RNA isolates of the same tissue type and replicated at least three times per preparation. RT-PCR products were analyzed semi-quantitatively by densitometry and compared as relative arbitrary units. The cycling steps recommended in the One Step RT-PCR manual were followed without modifications. The RT-PCR assays were conducted using 20 nanograms of total RNA and 30 cycles of amplification. The annealing temperature range used was from 55-65° C.

For DNA sequencing, select RT-PCR products were separated by polyacrylamide gel electrophoresis and purified by gel-extraction. The purified RT-PCR DNA products were each cloned using the pGEM-T-Easy TA-cloning kit (Promega, Madison, Wis.), screened, and then prepared for DNA sequencing using standard protocols.

The same approach and procedures were also used to analyze the structure of At1g25290 transcripts in Arabidopsis plants. This was reported in Sedivy-Haley et al., 2012.

### Example 3

### Construction of Bacterial Expression Plasmids for At1g74130 and Alternative Splice Variants

All DNA cloning and expression plasmids discussed were propagated in the Escherichia coli strains HB101 or the JM101-109 strain series. The transformation of various bacterial strains was carried out using standard protocols (such as the ones described in Molecular Cloning: A Laboratory Manual, Sambrook, et al. (1989) Cold Spring Harbor Laboratory, Cold Spring Harbor, N.Y.). Plasmid DNAs were isolated from the bacterial strains harboring the corresponding plasmids using standard protocols (such as the ones described in Molecular Cloning: A Laboratory Manual, Sambrook et al., 1989). DNA constructs that express the At1g74130 protein or its alternative splice variants can be created, inserted and propagated in a variety of noncommercial or commercially-available plasmids such as the pET series (EMD4 Biosciences (Novagen), Rockland, Mass., USA), the pBLUESCRIPT series (Stratagene), the pBS series (Stratagene), the pGEM and pSP series (Promega) and pT7/T3 series (Pharmacia) if the T7 RNA polymerase bacterial expression system is used to synthesize At1g74130 protein or its alternative splice variants. The T7 RNA polymerase gene in the appropriate bacterial strains such as JM109(DE3) or BL21(DE3) is under the control of the IPTG-inducible lac promoter. The currently used promoter for expression in the T7 RNA polymerase containing/expressing bacteria is the T7 promoter. Termination sequences such as the T7 terminator can be used in addition to any functionally equivalent sequences present in the gene itself. Other expression systems such as the IPTG-inducible system based on the lac promoter can also be used to express the At1g74130 protein or its alternative splice variants, for example, the pKK233 series (Clontech) or the pPROK series (Clontech). Any other bacterial expression system that causes the expression of the At1g74130 protein in a desirable manner including constitutive expression can also be used. Plasmids usually contain multiple cloning regions for cloning manipulations, an origin of replication and a selectable gene marker such as antibiotic resistance. Expression plasmids additionally contain an appropriate promoter.

All restriction endonuclease digestions were carried out in accordance with the buffers and protocols provided by the manufacturer of each particular enzyme. Restriction enzyme was added to give 5-10 units per microgram of DNA and the reaction mixture was adjusted to the appropriate final volume with water. The final volumes were usually 20-100 μl and contained 2-10 μg of plasmid DNA. Digestions were thoroughly mixed and carried out for 1 hour at the appropriate suggested temperature. Digested DNA molecules were re-purified by phenol and chloroform:iso-amyl alcohol extraction, centrifugation (usually in a microfuge) and the aqueous layer containing the digested DNA concentrated by precipitation in two volumes of 100% ethanol in the presence of 0.3M sodium acetate, pH 7.0 or 0.1M sodium chloride. The phenol used was saturated with 0.1M Tris-HCl pH 8.0 plus 0.1% (w/v) hydroquinoline prior to use. The chloroform:iso-amyl alcohol consisted of 24 volumes of chloroform and 1 volume of iso-amyl alcohol. Equal volumes of phenol or chloroform:iso-amyl alcohol were used in each of the organic solvent extraction steps. The DNA precipitates were collected by centrifugation, washed once with 70% ethanol (70% ethanol, 30% water), dried and redissolved in an appropriate volume of water prior to further manipulations.

The expression plasmids for At1g74130 and alternative splice variants were made by first retrieving the RT-PCR derived cDNA insert from the pGEM-T-Easy plasmid DNA described above. The RT-PCR procedure and amplification conditions used here were the same as that described in Example 2 above, when and where any needed optimizations were achieved. Retrieval from the pGEM-T-Easy plasmid DNA was achieved by NdeI and BamHI (or HindIII) restriction enzyme digestions. The NdeI and BamHI (of HindIII) restriction endonuclease sites were introduced and added via the DNA primers designed for the PCR amplification of the indicated cDNA sequence. The NdeI site was introduced to allow the use of the upstream 5′ ribosomal-binding sequence and in-frame translation. The BamHI (or HindIII) site was introduced to allow in-frame fusion to the Histidine tag at the carboxyl terminus. The purification of the cDNA insert was carried out using the standard low melting agarose gel and phenol extraction method (Molecular Cloning: A Laboratory Manual, Sambrook et al., (1989) Cold Spring Harbor Laboratory, Cold Spring Harbor, N.Y.). The low melting agarose was supplied by GIBCO-BRL, Gaithersburg, Md., U.S.A. DNA was recovered from appropriate low melting agarose slices by heating at 65° C. followed by extraction with phenol that had been prewarmed at 37° C. and centrifugation. The phenol extraction was repeated. The aqueous layer containing the DNA was then adjusted to 0.1M sodium chloride and centrifuged for 10 min in a microfuge. The supernatant was then given a chloroform:iso-amyl alcohol extraction followed by precipitation in ethanol as described above. The DNA pellet was then collected by centrifugation, washed with 70% ethanol, dried and resuspended in water. The pET20b plasmid was digested with the same enzymes and dephosphorylated. Phosphatase reactions were carried out by adjusting the restriction digestion reactions with 3.5 μl 1M Tris-HCl, pH 8.0 (per 100 μl reaction) and adding 0.5 unit of calf intestinal alkaline phosphatase. Incubation proceeded for 30 minutes at 37° C. and the DNA was then repurified by organic solvent extraction followed by ethanol precipitation as above.

The ligation reactions consisted of the two appropriate target DNA molecules, ligase buffer (50 mM Tris-HCl pH 7.5, 10 mM magnesium chloride, 1 mM dithiothreitol, 1 mM ATP) and 1-3 units of enzyme. The ligation reaction was carried out at 15° C. using T4 DNA ligase from various suppliers.

The same procedures and strategy disclosed above were used for the construction of bacterial expression plasmids for the other rhomboid protein variants, namely At1g25290 variants and UBAC2.

### Example 4

### Expression of At1g74130, At1g25290, and UBAC2 and Variants in E. coli

The expression plasmids for At1g74130 or its alternative splice variants were transformed into the E. coli strain JM109(DE3) using standard calcium chloride methods. Selected colonies were checked for the presence of the plasmid and its quantity in the cell. The resulting strains of JM109(DE3) containing these plasmids were recovered and stored in glycerol stocks at −70° C. until use. The glycerol stocks were made by taking 850 μl of log phase growing cells and mixing in 150 μl of sterile glycerol.

For each expression experiment, the cells were streaked out on LB-ampicillin plates (25 μg/ml) and incubated at 37° C. overnight. Colonies formed on the overnight plate were then used to inoculate a liquid culture of LB and ampicillin. Incubation again proceeded with shaking overnight at 37° C. This overnight liquid culture was then used to inoculate a culture to induce expression of At1g74130 variant proteins. The overnight liquid culture was used as an inoculum at a ratio of 1:100 and the freshly inoculated culture allowed to grow with shaking. The growth periods were modified to meet the purposes of the said experiments.

Induction of expression was achieved by the addition of isopropyl β-D thiogalactopyranoside (IPTG) (48 mg/ml stock) two hours after inoculation. The ratio of IPTG used for these inductions was 1000 μl per 500 ml culture. Expression was allowed to proceed as described below before using centrifugation to collect the cells for analysis or for purification in Example 5.

The pelleted cells used for analysis were resuspended in SDS-PAGE loading buffer (5% SDS (w/v), 0.1% bromophenol blue (w/v), 20% glycerol (v/v), 1.2M β-mercaptoethanol, 0.1M Tris-HCl pH 6.8), boiled for 1-3 minutes, and centrifuged full speed for 5-10 minutes in a microfuge before loading onto an SDS-polyacrylamide gel for analysis. Protein gels were visualized by Coomassie Blue staining. More specific protein bands were analyzed by immunoblotting using various specific antibodies as indicated accordingly in the various experiments.

The same procedures described above for At1g74130 variants were employed to produce equivalents for At1g25290 variants and UBAC2. The products were used either directly for analysis as described above, or for larger scale protein production described in below in Example 5.

### Example 5

### Recombinant Protein Production in E. coli

Recombinant proteins were produced in E. coli JM109 (DE3) cells. Rhomboid proteins for At1g74130 and the two At1g74130 splice variants were produced using the corresponding cDNA sequences (accession no. NM_202412) and the plasmid pET20b (ampicillin selection) (EMD4Biosciences (Novagen), Rockland, Mass., USA). The constructs were made with the addition of carboxyl-terminal histidine tags available through the pET20b plasmid. Although carboxyl-terminal histidine tags were used to facilitate purification, the recombinant proteins can be made without the addition of carboxyl-terminal histidine tags and purified through using other protocols or means. Histidine-tagged proteins were purified using nickel-nitriloacetic acid (Ni-NTA) affinity chromatography (Qiagen, Hilden, Germany). Known procedures were used for the cultivation of bacterial cells, the induction of protein expression, and affinity purification of histidine-tagged products. In the studies disclosed here, recombinant proteins were isolated using the materials, steps, components, concentrations, and buffers outlined in Lemberg, M. K., et al., Mechanism of intramembrane proteolysis investigated with purified rhomboid proteases, EMBO J. 24: 464-472, 2005. As described by Lemberg et al (2005), bacterial cells were grown at 16° C. in antibiotic-containing Terrific Broth (Bioshop Inc., Burlington, ON, Canada). Proteins were eluted initially in a buffer containing β-dodecyl maltoside, cholesteryl hemisuccinate (Anatrace; Affymetrix), imidazole, Hepes-NaOH, NaCl, CaCl₂, MgCl₂, and glycerol. β-dodecyl maltoside and cholesteryl hemisuccinate maintain the proteins in the functional state. The rhomboid proteins used for the assays were diluted at least to a final fold 23× in bacterial or yeast growth media. All purified protein samples were quantified using Bradford assay system and normalized before use.

The same procedure disclosed above for the production of At1g74130 variant proteins was employed to produce the At1g25290 and UBAC2 protein variants used in the examples below.

### Example 6

### Protein Gel and Immunoblotting Procedures

Recombinant protein samples, typically 0.5 μg, were loaded per lane in 1D 12% (w/v) denaturing SDS-polyacrylamide gels. Protein gel electrophoresis and immunoblotting were performed. The recipes and procedures accompanying the Biorad 1D/2D Minigel System (Biorad Inc, Hercules, Calif., USA) were used. Resulting immunoreactive bands were scanned, quantitated by densitometry, normalized, and compared relative to internal references when applicable. All immunoblots were replicated at least three times within each experiment and with independent experiments. Quantitations were conducted for each replicate using non-saturated scans of the results. All proteinaceous samples were resolved and analyzed by denaturing SDS-PAGE, electrophoretically transferred onto nitrocellulose filters and immunologically analyzed. Anti-rhomboid protein antibodies were tested against variant proteins and established using the same techniques as described here. Primary immunoreactions were detected using horse radish peroxidase-conjugated anti-rabbit IgGs (Sigma; Promega) and a chemiluminescent detection kit (e.g., Western Lightning PlusμECL kit from Perkin-Elmer, Waltham, Mass., USA). The resulting immunoblots were analyzed and normalized by densitometry using for example, ImageJ.

### Example 7

### Antibody Preparations

Monospecific type G immunoglobulins used in the various immunological experiments were prepared using female New Zealand white rabbits. Polyclonal antiserum or antibodies against the At1g74130 protein were generated. These proteins were generated using the T7 RNA polymerase-expressing bacterial overexpression system. The plasmid vector pET20b (EMD4Biosciences (Novagen), Rockland, Mass., USA) and E. coli strain JM109(DE3) were used to facilitate overexpression. Histidine-tagged proteins were purified using Ni-NTA affinity chromatography (Qiagen, Hilden, Germany) using the same procedure and materials described above in Example 5. Bacterial cells were grown at 16° C. in antibiotic-containing Terrific Broth (Bioshop Inc., Burlington, ON, Canada). All purified protein samples were quantified using Bradford assay system and normalized before use. Denatured proteins were prepared and purified by preparative SDS-PAGE prior to extraction and electroconcentration. Approximately 25 μg of antigens were used for each booster injection. Each injection in addition contained saline solution (150 mM sodium chloride and 10 mM phosphate buffer pH 7.0), 0.1% SDS (w/v) and TitreMax Gold adjuvant (CedarLanes Laboratories, Burlington, ON, Canada)). A total volume of 500 μl was injected each time. The initial immunization program occurred over a six-week period and the rabbits were given monthly boosters a week prior to bleeding. Preimmune sera and corresponding IgGs were collected and purified prior to the injection of each rabbit.

The antibodies were generated against recombinant proteins derived from the entire protein. The polyclonal anti-antibodies used were made in rabbits and were equally reactive for all forms, independent of mutations or synthesis source. Antibody specificity was established by control immunoblot assays using rabbit pre-immune serum and inherently through immunoblots employing other specific antibodies. Specificity was also witnessed using affinity-purified antibodies or affinity-purified recombinant At1g74130 proteins. Whole yeast cell or yeast mitochondria extracts from strains carrying plasmid only were also used to confirm specificity, i.e., no immunoreacting bands arose in the corresponding protein blots. The 1:1,000 dilution of anti-At1g74130 antibodies used was established by titration and linearity assessments. All immunoblots were repeated at least three times within each experiment and with independent experiments.

### Example 8

### Analysis of Changes in the Transport of β-Lactamase and Antibiotic Susceptibility

Changes to β-lactamase transport were monitored by two different approaches: 1) the ability to form colonies on solid LB-agar plates containing increasing concentrations of ampicillin (25 μg/ml to 2.0 mg/ml); and 2) the level of transported and processed β-lactamase detected by immunoblotting total protein samples of cells grown in LB-broth containing increasing ampicillin concentrations.

Expressing cells of At1g74130 or its alternative splice variants were grown independently in LB broth containing 25 μg/ml ampicillin to OD 600=0.8-1.0. Cultures were collected and optical density (OD) at 600 nm values measured to adjust by dilution the cell numbers (usually 500-800 cells) to approximately the same numbers before plating onto LB agar plates containing increasing amounts of ampicillin. Plates were incubated at appropriate temperatures overnight. Colony numbers on each plate with increasing ampicillin concentrations were compiled and compared to assess the level of ampicillin resistance based on the ability to form colonies. Morphological aspects of the formed colonies, i.e., growth characteristics, were documented by digital imaging.

The same cells were also used to determine the level of transported β-lactamase grown in media containing increasing concentrations of ampicillin. Cultures were diluted 50 times into LB broth with higher amounts of ampicillin (1.5 mg/ml and 1.75 mg/ml) and allowed to grow up overnight. The level of transported β-lactamase was monitored by immunoblotting analysis of samples taken after an overnight incubation or growth. Immunoblot analysis was performed on total cellular protein samples with anti-β-lactamase antibodies as outlined above.

### Example 9

### Sensitization of Bacteria to Ampicillin

Experiments were conducted to assess the ability of At1g74130 proteins or variants to sensitize bacteria to the antibiotic ampicillin, using “super-resistant” E. coli (multi-copy pET20b plasmid-based ampicillin-resistant lab strains). This model is considered the so-called “SuperBug” model, as commonly referred to in the public domain and media.

Control assays were conducted on cells without added At1g74130 or variants (sensitizing variants) to determine the delivery system to use and the appropriate levels of ampicillin to use. Control assays were also conducted to determine if the 5% (v/v) DMSO delivery system exerted any effects. Bacterial cells, usually 800-1000, were mock treated with Luria Broth (LB) media or 5% (v/v) DMSO for 15 minutes prior to exposure to a 30-45 minute exposure to ampicillin (1.25 mg/ml or 1.5 mg/ml). Total volumes of 200 μl were used in these assays. The treated-exposed cells were then plated onto non-selective LB agar plates and allowed to grow overnight to assess the level of cell death or susceptibility to ampicillin. The ampicillin range found useful for these assays was between 1.25 mg/ml to 1.5 mg/ml. This range mirrored the observations reported in an earlier section above with transgenic bacterial cells and ampicillin susceptibility.

In the experimental assays, the sensitizing properties of At1g74130 or variants (sensitizing variants) were tested by adding 10 ug proteins (At1g74130 or variants) and 5%( v/v) DMSO to bacterial cells (800-1000), incubated for 30-45 minutes, and followed by the 30-45 minute treatment with 1.25 mg/ml or 1.5 mg/ml ampicillin. The treated cells were then plated on non-selective LB plates to assess susceptibility to ampicillin, by documenting the level of cell growth impairment and cell death, i.e., the number of cells left live after treatment. Morphological aspects of the formed colonies, i.e., growth characteristics, were documented by digital imaging.

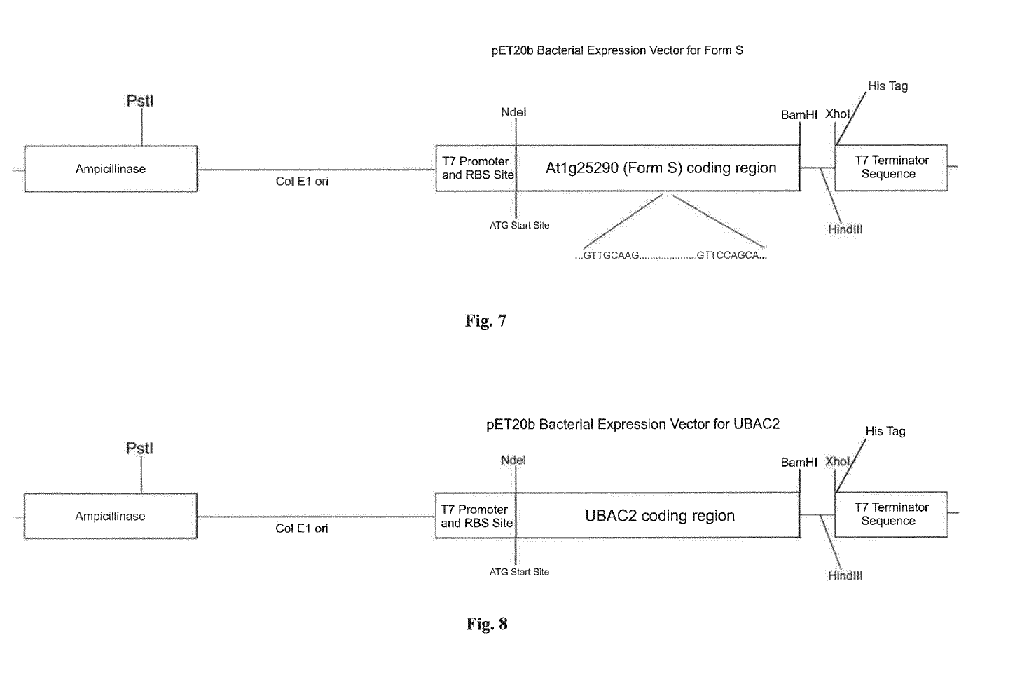

In the results presented in FIG. 12, “No treatment” cells were treated with 1.25 mg/mL ampicillin only. “L with DMSO” cells were treated with 10 μg of At1g74130 (splice variant L), 5% DMSO and 1.25 mg/mL ampicillin. “M with DMSO” cells were treated with 10 μg of At1g74130 (splice variant M), 5% DMSO and 1.25 mg/mL ampicillin. “S with DMSO” cells were treated with 10 μg of At1g74130 (splice variant S), 5% DMSO and 1.25 mg/mL ampicillin. “Mock treatment” cells were treated with 5% DMSO, adjusted elution buffer, and 1.25 mg/mL ampicillin. The results show that all three At1g74130 splice variants reduced survival of ampicillin resistant E. coli. The assays were carried out in triplicate and exhibit statistical significance. Compared to the control, the mock treatments did not differ significantly (T-test, P=0.36). Compared to mock treatments, assays containing L, M, and S were significantly different (T-test, P=0.022; P=0.026; and P=0.029, respectively). Exogenous application of the At1g74130 or variants (sensitizing variants) increased the sensitivity of bacteria to ampicillin when co-delivered with the At1g74130 or variants. The effective co-delivery concentrations of ampicillin or protein, with or without a delivery system, had no effects when provided individually. The delivery system (5% (v/v) dimethyl sulfoxide (DMSO)) also had minimal effect on the susceptibility of the bacterial cells to ampicillin. Only the combination of ampicillin and At1g74130 or variants (sensitizing variants) in 5% (v/v) DMSO resulted in an impairment in cell growth and cell death (in the range of 20-40% relative to control or mock treatment assays).

### Example 10

### Construction of Yeast Expression Plasmids for At1g74130 and Alternative Splice Variants

The expression of proteins in yeast was accomplished by using two yeast—E. coli shuttle vectors, YEplac195 or pACT2 (Clontech Laboratories, MountainView, Calif., USA). The production of At1g74130 or equivalents was achieved by inserting the corresponding cDNA fragments into YEplac195 or pACT2 (see FIGS. 9, 10, and 11). Gene expression was facilitated using either the constitutive promoter (Adh) derived from the yeast alcohol dehydrogenase gene or the developmentally-regulated yeast promoter (Rbd1) of the endogenous RBD1 (PARL) mitochondrial rhomboid protease. These two alternative splice variants were derived by RT-PCR cloning and tested along with the full version of At1g74130, which was also derived by RT-PCR cloning, as opposed to being obtained from resources available to users, such as the Arabidopsis Resources available at the Ohio State University and its consortium. The alternative splice variants designated At1g74130-7/8 and At1g74130-6/7 and are shown in FIGS. 1C and 1D, respectively. The two variants tested were the same ones tested in bacteria (described above). All genes and related constructs were confirmed by DNA sequencing. The introduction of plasmids into yeast cells was carried out using standard yeast transformation techniques. The wild type yeast strain used was acquired from EUROSCARF. All experiments were conducted with cells grown in glucose-supplemented media (propagated at 27-30° C. in glucose-supplemented YC medium without uracil or without uracil and leucine). All strains were prepared and stored at −80° C. as glycerol stocks.

All restriction endonuclease digestions were carried out in accordance with the buffers and protocols provided by the manufacturer of each particular enzyme. Restriction enzyme was added to give 5-10 units per microgram of DNA and the reaction mixture was adjusted to the appropriate final volume with water. The final volumes were usually 20-100 μl and contained 2-10 μg of plasmid DNA. Digestions were thoroughly mixed and carried out for 1 hour at the appropriate suggested temperature. Digested DNA molecules were re-purified by phenol and chloroform:iso-amyl alcohol extraction, centrifugation (usually in a microfuge) and the aqueous layer containing the digested DNA concentrated by precipitation in two volumes of 100% ethanol in the presence of 0.3M sodium acetate, pH 7.0 or 0.1M sodium chloride. The phenol used was saturated with 0.1M Tris-HCl pH 8.0 plus 0.1% (w/v) hydroxquinoline prior to use. The chloroform:iso-amyl alcohol consisted of 24 volumes of chloroform and 1 volume of iso-amyl alcohol. Equal volumes of phenol or chloroform:iso-amyl alcohol were used in each of the organic solvent extraction steps. The DNA precipitates were collected by centrifugation, washed once with 70% ethanol (70% ethanol, 30% water), dried and redissolved in an appropriate volume of water prior to further manipulations.

The yeast expression plasmids At1g74130 and its alternative splice variants were made by using the cDNA inserts from the recombinant DNA plasmids described above for the bacterial experiments. Here, to facilitate transfer and insertion into the pACT2-based yeast expression plasmids, the bacterial expression plasmids containing cDNA sequences encoding At1g74130 or variants were used as templates for PCR amplification. The DNA primers used were designed to incorporate suitable restriction enzyme sites required for insertion into the pACT2-based yeast expression plasmids. PCR amplified DNA sequences were either used directly for subcloning or subcloned first into pGEM-T-Easy like that described above, isolated, purified and then retrieved from the recombinant pGEM-T-Easy plasmids using a suitable pair of restriction enzymes. In this case, the restriction endonuclease pair used consisted of SacI and SacII. Retrieval was thus achieved by SacI and SacII restriction enzyme digestions. The purification of the SacI-SacII cDNA inserts was carried out using the standard low melting agarose gel and phenol extraction method. The low melting agarose was supplied by GIBCO-BRL, Gaithersburg, Md., U.S.A, or other companies selling the same products. DNA was recovered from appropriate low melting agarose slices by heating at 65° C. followed by extraction with phenol that had been prewarmed at 37° C. and centrifugation. The phenol extraction was repeated. The aqueous layer containing the DNA was then adjusted to 0.1M sodium chloride and centrifuged for 10 min in a microfuge. The supernatant was then given a chloroform:iso-amyl alcohol extraction followed by precipitation in ethanol as described above. The DNA pellet was then collected by centrifugation, washed with 70% ethanol, dried and resuspended in water. The pACT2-basedyeast expression plasmids were digested with the same enzymes and dephosphorylated. Phosphatase reactions were carried out by adjusting the restriction digestion reactions with 3.5 μl 1M Tris-HCl, pH 8.0 (per 100 μl reaction) and adding 0.5 unit of calf intestinal alkaline phosphatase. Incubation proceeded for 30 minutes at 37° C. and the DNA was then repurified by organic solvent extraction followed by ethanol precipitation as above.

The ligation reactions consisted of the two appropriate target DNA molecules, ligase buffer (50 mM Tris-HCl pH 7.5, 10 mM magnesium chloride, 1 mM dithiothreitol, 1 mM ATP) and 1-3 units of enzyme. The ligation reaction was carried out at 15° C. using T4 DNA ligase from various suppliers.

Insertion of the SacII-SacI cDNA fragments into pACT2 occurred between the promoter (either Adh or Rbd1) and ADH1 terminator. The pACT2-based vector containing the Rbd1 promoter was modified to allow the insertion of the Rbd1 promoter sequence. This modification was facilitated by mutating the resident NdeI restriction enzyme site in pACT2 so that this site is no longer recognized by NdeI. This particular NdeI site was located on the plasmid backbone. The other NdeI site near the 5′ end of the Adh promoter was used for replacing the resident Adh promoter with the Rbd1 promoter. The Rbd1 promoter, an NdeI-(newly introduced SacII)-BamHI DNA fragment, was inserted into pACT2 using the restriction endonuclease sites NdeI and BamHI. The Rbd1 promoter sequence was cloned by PCR amplification of yeast genomic DNA and inserted into the pGEM-T-Easy vector so that the promoter sequence can be retrieved as an NdeI-BamHI DNA fragment for insertion into the NdeI modified pACT2 vector. The SacII site was introduced to facilitate subsequent insertion of At1g74130 and variants downstream.

### Example 11

### Expression of At1g74130 and Alternative Splice Variants in Saccharomyces cerevisiae

The yeast expression plasmids for At1g74130 or its alternative splice variants were transformed into Saccharomyces cerevisiae using standard chemical methods, such as lithium acetate and polyethylene glycol. Selected colonies were checked for the presence of the plasmid and its quantity in the cell. The resulting strains containing these plasmids were recovered and stored in glycerol stocks at −70° C. until use. The glycerol stocks were made by taking 850 μl of log phase growing cells and mixing in 150 μl of sterile glycerol.

For each expression experiment, the cells were streaked out on selective plates and incubated at 27-30° C. for 2-3 days. Colonies formed on the plate were then used to inoculate a liquid culture of the same selection media. Incubation again proceeded with shaking overnight at 27-30° C. This overnight liquid culture was then used to inoculate a culture to allow expression of At1g74130 or its related/equivalent proteins. The overnight liquid culture was used as an inoculum at a ratio of 1/100 and the freshly inoculated culture allowed to grow with shaking.

The pelleted cells were then processed according to the experimental requirements. Resulting preparations from whole cell protein extracts to mitochondrial isolations were typically resuspended in SDS-PAGE loading buffer (5% SDS (w/v), 0.1% bromophenol blue (w/v), 20% glycerol (v/v), 1.2M 3-mercaptoethanol, 0.1M Tris-HCl pH 6.8), boiled for 3 minutes, and centrifuged for 5 minutes in a microfuge before loading onto an SDS-polyacrylamide gel for analysis. Protein gels were visualized by Coomassie Blue staining or more specific protein bands were analyzed by immunoblotting with various specific antibodies as described above.

### Example 12

### Analysis of Changes in the Yeast Mitochondrial System Using Immunoblots

For yeast strains studied here, mitochondria were isolated and samples were quantitated using the Bradford assay system (purchased from Biorad, Hercules, Calif.) and normalized prior to immunological analysis. Standard protein blotting techniques were used. The immunoblot results were analyzed by densitometry when applicable. Immunoreactive bands were scanned, quantified, and assessed relative to each other. The use of antibodies was pre-determined by titration assays in previous work. Control experiments were performed to determine the linearity of band signals for the antibodies used and the proteins being assessed. Scans were conducted for each replicate using non-saturated versions of the results presented. Changes in the band patterns were assessed by measuring the intensities of the protein bands. Ratios were used to quantify changes to the band profiles. Three immunoblots were analyzed for each experiment as well-as from independent repeat experiments. Each band on each of the individual blots was further quantified three times.

### Example 13

### Sensitization of Yeast to Fungicidal Agents Using At1g74130 Variants

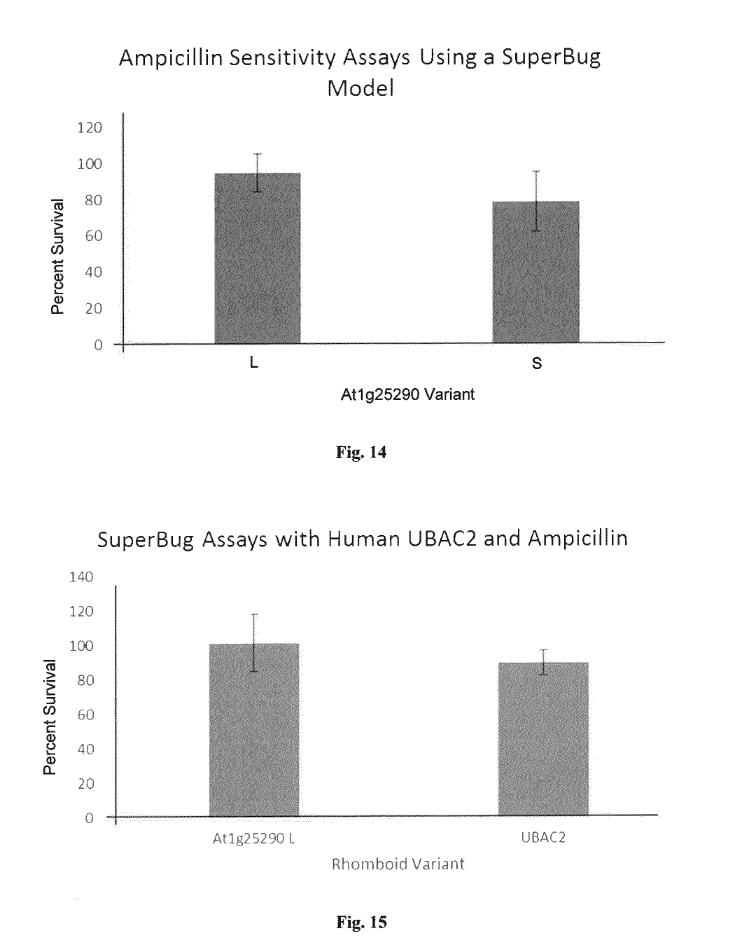

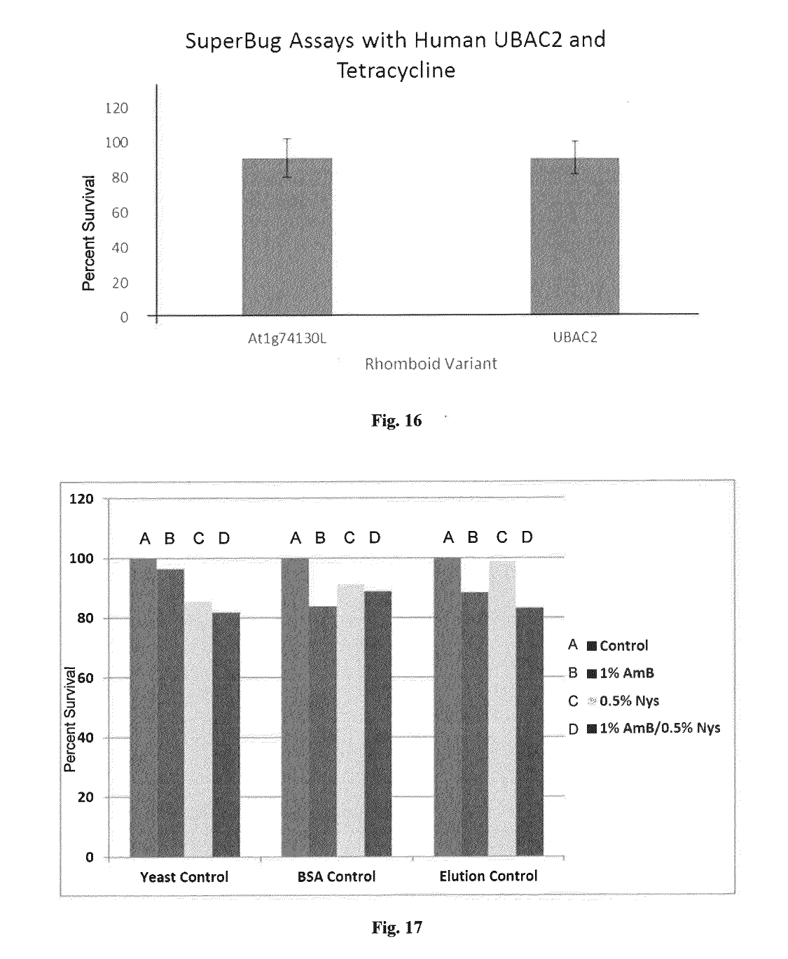

Experiments were conducted to assess the ability of the Arabidopsis At1g74130 proteins or variants to sensitize yeast to the antifungal agents Amphotericin B (AmB) and Nystatin (Nys). Overall, these assays were conducted in a manner similar to that designed for bacteria (Example 9). In addition to the components mixed with the added proteins or variants, the antifungal AmB was also utilized as part of the delivery system. The AmB drug contains small quantities of the detergent deoxycholate, which may also assist in the delivery of proteins into the cell, as well as function as a pore forming agent itself. In this setting, the results indicate that exogenous application of the At1g74130 or variants (sensitizer variants) increased the sensitivity of yeast to sub-lethal levels of antifungals when co-delivered with the At1g74130 or variants (sensitizer variants). The effective co-delivery sub-lethal concentrations of the two antifungal agents or protein had little or no effect when provided individually. FIGS. 17 and 18 show results of the yeast sensitivity assays for AmB and Nys. Based on the levels used for this study, the results demonstrate that relative to the experimental assays, little to no effects were observed with culture media (YPD), AmB, Nys, or the combination of AmB and Nys, but no rhomboid proteins. In FIG. 17, the “Yeast Control” bars represent yeast cells given mock treatments with culture media (YPD) alone, YPD plus 1% AmB, YPD plus 0.5% Nys, and YPD plus 1% AmB and 0.5% Nys, respectively. The “BSA Control” bars represent another set of mock treatments with yeast cells. This set utilized the same set-up as the Yeast Control set but with added control bovine serum albumin proteins (BSA) (dissolved in the elution buffer, the same buffer used for the purification of the recombinant At1g74130 rhomboid proteins or variants). These assays represent another variation of negative controls for rhomboid protein. The “Elution Control” bars represent yeast cells mock treated using the same set-up as in the Yeast Control set but with added elution buffer alone. This variation of control assays was designed to assess for potential toxic impacts when combining elution buffer and the listed drugs, as well any effects originating from components of the elution buffer itself. The composition of the elution buffer is provided above in Example 5. With the exception of Nys, these treatments represent the first step and occur for 45 minutes before the second step when exposure to fungicides takes place. Within each set of bars, where applicable, the second 15-minute step treatment occurred with a sub-lethal dose of Nys. After a total of one hour, the treated yeast cells were plated on non-selective YPD agar plates. Plates were grown 2-3 days at 27-30° C. and assessed for surviving cell numbers (measuring cell death). Images were also taken to document cell and colony growth characteristics.

FIG. 18 shows results of treatments, starting with control assays (the same as the ones shown in FIG. 17) and various combinations with or without the At1g74130 L, M, and S rhomboid variant proteins. Treatments with sub-lethal levels of AmB, Nys, and AmB/Nys alone caused a small reduction in yeast cell survival (columns designated in FIG. 18 as None, AmB, Nys, and Amb/Nys). All three variants had no effect on yeast cell survival when each was added alone in elution buffer (columns designated as L alone, M alone, and S alone). However, treatments with the fungicidal agents together with the At1g74130 L, M, or S variant proteins dramatically reduced yeast survival (the remaining six columns in FIG. 18).

In the experimental assays, the sensitizing properties of At1g74130 or variants (sensitizing variants) were tested by adding 10 ug protein (At1g74130 or variants in elution buffer) to yeast cells (300-500 cells). To reiterate in all cases, the components were added to cultures and mixed for one hour, where Nys was added and incubated for the last 15 minutes of the treatment. Pre-treatment was designed to permit AmB and At1g74130 proteins to establish and work prior to adding Nys. After the one hour incubation, 500 cells were plated on non-selective YPD agar media and grown for 48 hours at 30′C.

The sub-lethal levels of fungicides to use in these assays were determined using dosage experiments. These assays were conducted on yeast cells without added rhomboid proteins or variants. The dosages of AmB tested ranged from 0 to 5% (v/v). As there were no significant differences in surviving yeast colonies within the range of 0 to 1%, 1% was used for optimization with rhomboid protein assays. Likewise, the Nys level was determined using a range of 0.5 to 2.5%. Yeast cells were treated for one hour with fungicides in a total volume of 400 μl YPD media before plating.

Since both the AmB buffer contained small quantities of detergent (deoxycholate being the main one), an experiment was done in which different amounts (1% and 10%) of deoxycholate were added to yeast cells to ascertain any effects. Adding deoxycholate had little effect on yeast.

### Example 14

### Sensitization of Bacteria to Tetracycline Using At1g74130 Variant Proteins

Experiments were conducted to assess the ability and the utility of At1g74130 protein variants to sensitize bacteria to another antibiotic, tetracycline. In bacteria, tolerance or resistance to tetracycline is facilitated by a mechanism different from the ampicillin resistance assayed in Example 9. Unlike the breakdown of ampicillin outside the bacterial cells by secreted ampicilliase, tetracycline resistance in bacteria is based largely on the continual and effective pumping of tetracycline out of the cell by proteinaceous membrane pumps. To assess the utility of the At1g74130 for sensitizing bacteria to tetracycline, these bacterial assays were conducted in the same manner described in Example 9. The same controls and combinations were used. The final treatment level of tetracycline used in place of the ampicillin treatment was 15 ug/ml.

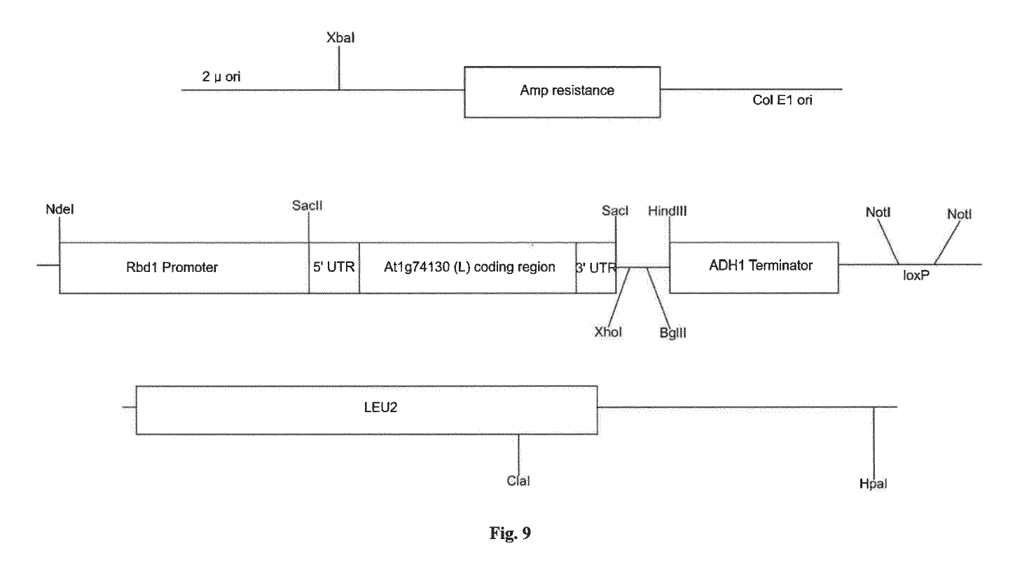

Similar behavior and responses were observed with At1g74130 variant proteins and tetracycline sensitivity, albeit at a level different from that observed for ampicillin (FIG. 13). Sensitizing effects were most obvious using the M form of At1g74130. The differences in the level of sensitivity displayed, for example, between variant forms of rhomboids or between different mechanisms for antibiotic resistance, could be related to the application context/setting in which the rhomboid proteins were applied, i.e., impacting established drug pumps versus protein secretion, and/or structural features of the variant proteins used. As with the At1g74130 bacterial assays above for ampicillin, exogenous application of the At1g74130 variant M increased the sensitivity of bacteria to tetracycline. The assays for L, M, and S were significantly different (n=4, T-test, p=0.01). Again, the different level of sensitivity displayed by variant M, as opposed to L or S, suggests that the phenomenon observed in these assays is attributed to the added rhomboid protein variant and not to other components in the mixtures. In effect, the testing of different rhomboid protein variants, such as L versus M versus S forms of At1g74130 and displaying sensitivity or not, acts as ultimate controls, i.e., equivalent to controls using protein counterparts with no known activity. In the case of At1g74130, variant L or S was acting in a capacity of the ultimate control, i.e., a protein counterpart with no activity.

### Example 15

### Sensitization of Bacteria to Ampicillin Using Rhomboid Protein Variants Derived from At1g25290

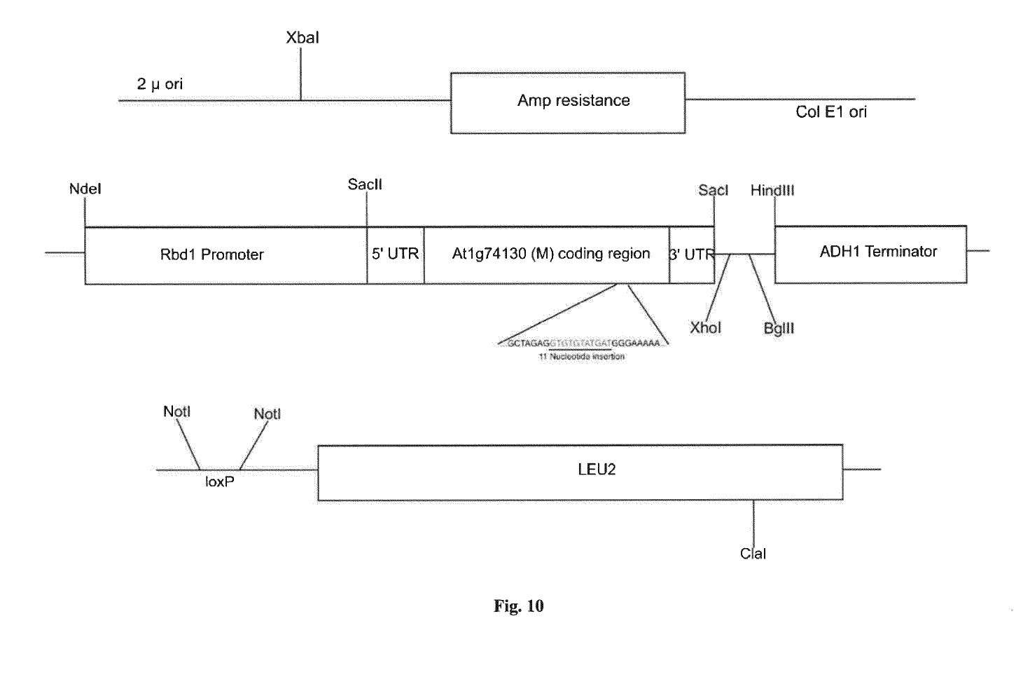

Experiments were conducted to assess the ability and the utility of other rhomboid types or variants to sensitize bacteria to ampicillin. These proteins were L and S variant forms of Arabidopsis At1g25290 (classified under the category of “Active Rhomboid Proteases”) and designated NM_102339.3 and NM-0011981641.1, respectively (Sedivy-Haley et al., 2012). The difference between the L and S forms is the presence or absence of a short RVL putative cyclin-binding domain. This three amino acid residue RVL domain is linked to cell cycle control and cell growth, such as in cancer cells. Variant L contains the RVL cyclin-binding domain, whereas variant S lacks these the three amino acid residues. To assess the utility of the At1g25290 variants for sensitizing bacteria to ampicillin, the bacterial assays were conducted in the same manner described for At1g74130 in Example 9. The same controls and combinations were used. Similar behaviours and responses were observed with the variant S form of At1g25290, albeit at a different level from that observed with the At1g74130 protein variants (FIG. 14). The assays for L and S were different (n=4, T-test, p=0.1). Again, the different level of sensitivity displayed by variant S, as opposed to L, suggests that the phenomenon observed in these assays is attributed to the added rhomboid protein variant and not to other components in the mixtures. In effect, the testing of different rhomboid protein variants, such as L versus S forms of At1g25290 and displaying resulting sensitivity or not, acts as ultimate controls, i.e., equivalent to controls using protein counterparts with no known activity. In the case of At1g25290, variant L was acting in a capacity of the ultimate control, i.e., a protein counterpart with no activity.

### Example 16

### Sensitization of Bacteria to Ampicillin Using a Human Rhomboid Protein Variant Derived from UBAC2

Experiments were conducted to assess the ability and the utility of a human rhomboid type or variant to sensitize bacteria to ampicillin. The human UBAC2 variant is a fusion between a rhomboid protein sequence (considered a rhomboid pseudoprotease) and ubiquitin-associating domains (gene ID 337827; XM_006719947.1; NM_001144072.1; Christianson, J. C., et al., Defining human ERAD networks through an integrative mapping strategy, Nature Cell. Biol. 14:93-105, 2012; Sawalha, A. H., et al., A putative functional variant within the UBAC2 gene is associated with increased risk of Behçet's disease, Arthritis and Rheumatism 63:3607-3612, 2011).

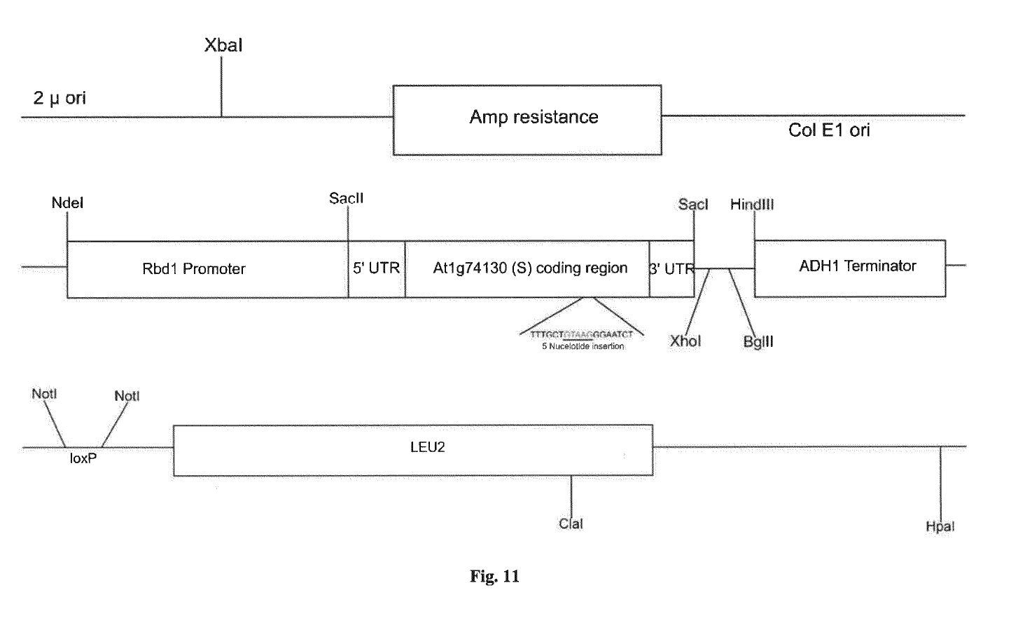

To assess the utility of the UBAC2 for sensitizing bacteria to ampicillin, these bacterial assays were conducted in the same manner described above for the other rhomboid variants and in Example 9. The same controls and combinations were used. The At1g25290 L was the control for UBAC2 in this set of assays. Similarly, the previously observed behaviours and responses were observed with UBAC2, albeit at a different level from that observed with the other variant proteins tested (FIG. 15).

### Example 17

### Sensitization of Bacteria to Tetracycline Using a Human Rhomboid Protein Variant Derived from UBAC2

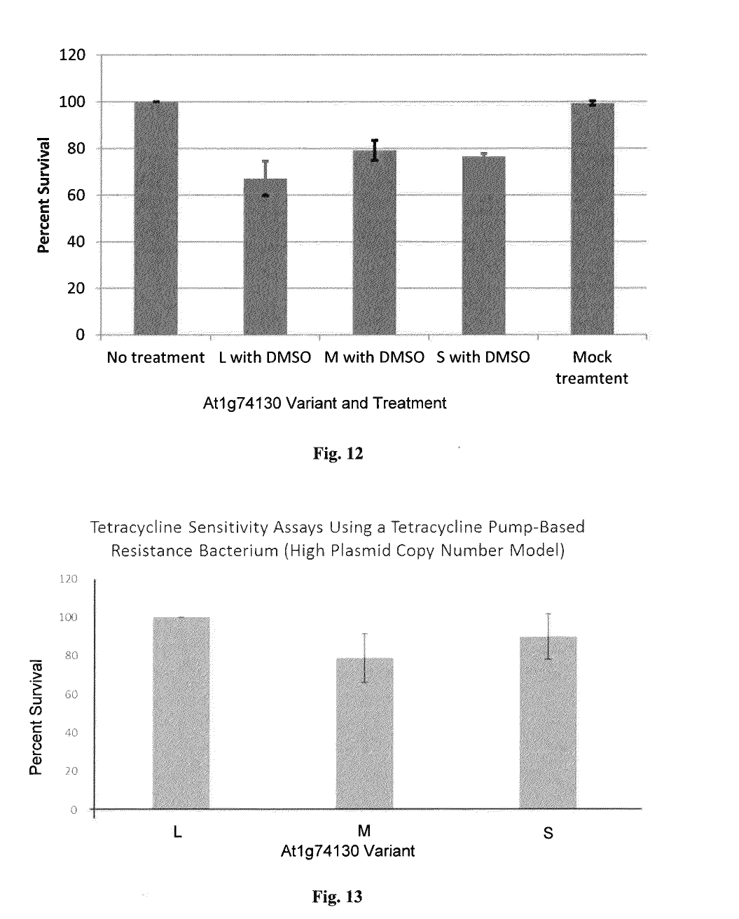

Experiments were conducted to assess the ability and the utility of a human rhomboid type or variant to sensitize bacteria to tetracycline. These bacterial assays were conducted in the same manner described above for the other rhomboid variants and in Example 9. The same controls and combinations were used. The At1g74130 L was the control for UBAC2 in this set of assays. The previously observed behaviours and responses were not observed for UBAC2 in this setting with the antibiotic tetracycline (FIG. 16).

### Example 18

### Sensitization of Yeast to Antifungals Using Rhomboid Protein Variants Derived from At1g25290

Experiments were conducted to assess the ability and the utility of other rhomboid types or variants to sensitize yeast to the antifungal agents AmB and Nys. Like for bacteria, the other variants tested were two Arabidopsis At1g25290 variants (L and S variant forms of At1g25290 classified under the category “Active Rhomboid Protease, i.e., variants NM_102339.3; NM-0011981641.1; Sedivy-Haley et al., 2012). As noted above, the difference between the two At1g25290 variants, L and S, is the presence or absence of a short RVL putative cyclin-binding domain. This RVL domain is linked to cell cycle control and cell growth, such as in cancer cells. Variant L contains the RVL cyclin-binding domain, whereas variant S lacks these three amino acid residues.

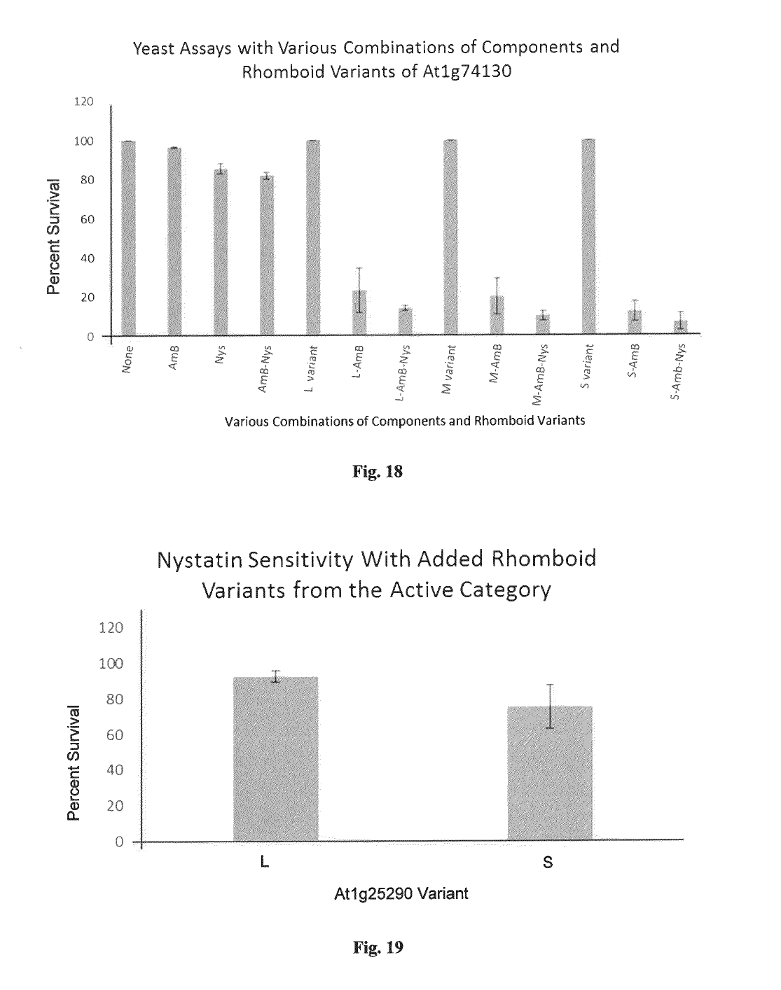

This set of yeast assays was conducted in the same manner described above (Example 13) and employed the same controls and combinations. Similar behaviours and responses were observed with the variant S form of At1g25290, albeit at a different level (FIG. 19). The assays for At1g25290 L and S were significantly different (n=4, T-test, p=0.01). Again, the different level of sensitivity displayed by variant S, as opposed to L, suggests that the phenomenon observed in these assays is attributed to the added rhomboid protein variant and not to other components in the mixtures. In effect, the testing of different rhomboid protein variants, such as L versus S forms of At1g25290 and displaying resulting sensitivity or not, acts as ultimate controls, i.e., equivalent to controls using protein counterparts with no known activity. In the case of At1g25290, variant L was acting in such a capacity, i.e. a protein counterpart with no activity.

### Example 19

### Sensitization of Yeast to Antifungals Using a Human Rhomboid Protein Variant Derived from UBAC2

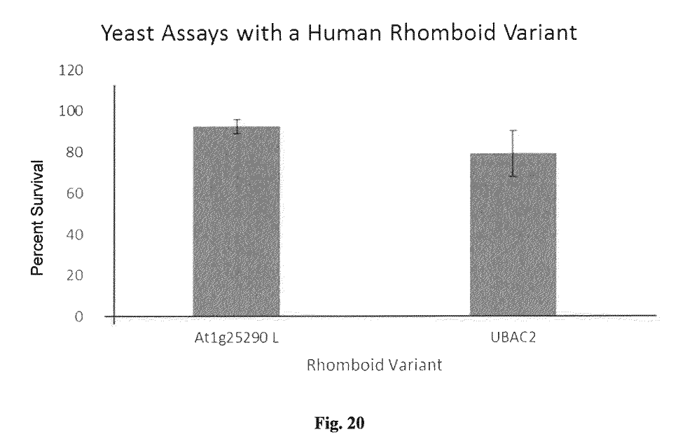

Experiments were conducted to assess the ability and the utility of mammalian rhomboid types or variants to sensitize yeast to the antifungal agents AmB and Nys, using human UBAC2 variant (isoform 1) as described above (see Example 16). This set of yeast assays was conducted in the same manner described above (Example 13) and employed the same controls and combinations. The At1g25290 L variant was the control for UBAC2. Similar behaviors and responses were observed with the human UBAC2 variant protein, albeit at a different level (FIG. 20). The assays for At1g25290 L and UBAC2 were different (n=4, T-test, p=0.01). The differences in the level of sensitivity displayed may be related to the application context/setting and/or structural features of the variant proteins used. As with the other yeast assays above, exogenous application of the UBAC2 variant proteins increased the sensitivity of yeast to sub-lethal levels of antifungals when co-delivered. As before, the effective sub-lethal concentrations of the two antifungal agents or protein used in these co-delivery assays had little to no effects when applied individually.

### Example 20

### Impairment of Lipase Secretion in Pseudomonas aeruginosa and the Bacterium's Subsequent Utilization of Oil as an External Carbon Source

The collective results from the bacterial and yeast assays with the different variants of rhomboids suggest that there may be other useful applications of the rhomboid proteins or variants. Experiments were thus conducted to assess the ability of At1g74130 proteins or variants to impair the protein secretion process of Pseudomonas aeruginosa, a gram-negative bacterium known to be pathogenic and producers of biofilms. Any impact on the protein secretion mechanism may alter pathogenicity, growth, survival, biofilm production, and sensitivity to antimicrobial drugs. The design of the Pseudomonas assays was similar to that used for bacteria in the above examples, results, and figures. Treated Pseudomonas cells were plated onto minimal media supplemented with olive oil as the only external carbon source. The minimal media-olive oil plates consisted of the following: 50 mM MOPS, pH 7.5; 40 mM K2HPO4; 25 mM NaH2PO4; 7.5 mM (NH4)2SO4; 0.4 mM MgSO4; 0.5% (v/v) olive oil; 40 ug/ml neutral red; 20 ul trace elements (Mn, Co, Zn, Cu, and Mo); and 1.5% agar. Pseudomonas cells need to express and secrete lipases in order to break down the olive oil and allow the acquisition of carbon from the media. Cells that are impaired with respect to protein secretion will not be able to break down the olive oil for acquisition and permit subsequent growth. This results in no cell or colony growth—the indicator used in these assays testing the utility of rhomboid proteins in the Pseudomonas setting.

Control assays were conducted on cells without added At1g74130 or variants (sensitizer variants) to determine the delivery system to use. Control assays were also conducted to determine if the 5% (v/v) DMSO delivery system used for E. coli exerted any effect. Pseudomonas cells were mock treated with Luria Broth (LB) media or 5% (v/v) DMSO for 45-90 minutes prior to plating on minimal media supplemented with olive oil as the sole external carbon source. Total volumes of 200 μl were used in these assays. The plates were allowed to grow overnight to assess the level of growth or colony formation. The results of these DMSO assays indicated that the impact of DMSO was little to none, confirming that it can be used as a delivery system for Pseudomonas.

In the experimental assays, the utility of At1g74130 or variants in the Pseudomonas was tested by adding 10 ug protein (At1g74130 or variants) to cells (600-800 cells) incubated for 45 minutes at 37° C., shaken, and followed by another 45 minute period at 37° C. The testing of the At1g74130 variant proteins was conducted with or without 5% (v/v) DMSO. The treated cells were then plated to assess the level of cell or colony growth impairment, i.e., the number of colonies appearing.

The results demonstrated that both At1g74130 splice variants M and S exhibited reduced, albeit different levels, of growth on the minimal media-olive oil agar plates, indicating impairment of the protein secretion system and hence the ability to break down and utilize olive oil as a carbon source. Of the three At1g74130 variant proteins tested, variant S displayed the most potent impact on the growth of cells (impairing lipase secretion) and hence less likely to advance to significant biofilm formation. Variants L and M may be viewed as proteins with no known activities as controls for variant S.

### Example 21

### Enhancement of Peptide Delivery with Added Rhomboid Protein Variants in Pseudomonas aeruginosa

With the Pseudomonas assay system described above, it was also possible to assess the ability and the utility of At1g74130 protein variants in the delivery of other peptides into cells. The assays were designed in the same manner as above to determine if peptide delivery can be enhanced or facilitated by rhomboids. The other peptides used were designed to inhibit protein secretion, in this case, lipases. The peptides used in these assays were nine amino acids long and targeted a specific region of one protein subunit of the Type II secretion machinery pseudopilin. The inhibitory actions of this nine amino acid peptide are due to interactions with the target region of the protein subunit. If peptide delivery was facilitated or enhanced by rhomboids, outcomes may be observed by changes in cell/colony growth (Pseudomonas) on the minimal media-olive oil plates. Such changes in growth would reflect changes in lipase secretion, which is needed for breaking down olive oil so that the released carbon can be used for cell growth.

Peptides designed for these types of actions or uses (such as that described in this example) are commonly between 6 and 20 amino acid residues in length. Such peptide approaches are reported for other systems, for example, in a viral setting (Hernaez, B., et al., Small peptide inhibitors disrupt a high-affinity interaction between cytoplasmic dynein and a viral cargo protein, J. Virol. 84:10792-10801, 2010), in a eukaryotic cell setting (Pazgier, M., et al., Structural basis for high-affinity peptide inhibition of p53 interactions with MDM2 and MDMX, Proc. Natl. Acad. Sci. U.S.A. 106:4665-4670, 2009), or in a bacterial-host setting (Bartels, M., et al., Peptide-mediated disruption of NFkappaB/NRF interaction inhibits IL-8 gene activation by IL-1 or Helicobacter pylori, J. Immunol. 179:7605-7613, 2007).

The results show that the At1g74130 M form exhibited growth characteristics suggesting the facilitation and/or enhancement of peptide delivery by the presence of rhomboid proteins. Depending on the combination of rhomboid and peptide, the ability of the Pseudomonas cells to grow, or not, was influenced by the presence of rhomboids and peptides. In some combinations, peptide delivery resulted in a competition that impacted the bacteria's ability to use olive oil as a carbon source. These patterns were only plausible if peptide delivery was made possible and made more efficient by the exogenously added rhomboid proteins.

The addition of the M variant of At1g74130 resulted in moderate impairment of lipase secretion (compared to the S variant above in Example 20) and less growth without the full ability to acquire carbon (i.e., the assay with elution buffer and M). The delivery of the M variant further into the cell releases the impairment and allows growth to resume (i.e., the assay with elution buffer, DMSO, and M). The addition of peptides in this case appears to impact secretion of lipase and reduce growth in the presence of the M variant and DMSO (i.e., the assay with peptides, elution buffer, DMSO, and variant M added tandemly). The impact of the peptides was greater when peptides were pre-incubated with the M variant before adding this mixture to the assay (i.e., the assay with peptides and M pre-incubated for 45 minutes, elution buffer, and DMSO). This outcome provides evidence as to where the M variant may be acting: the protein secretion machinery of the pseudophilin in the intermembrane/outer membrane location. This result also provides an indication that the M variant enhances the delivery of the peptide into the cell.

### Example 22

### Sensitization of Human Breast Cancer Cells to the Chemotherapeutic Drug Lapatinib Using At1g74130

The collective results from the bacteria (E. coli and Pseudomonas) and yeast assays with the different variants of rhomboids suggest that there may be further useful applications of the rhomboids. Experiments were thus conducted to assess the ability of At1g74130 proteins or variants to enhance the sensitivity of two breast cancer cell lines to the chemotherapeutic drug lapatinib. The breast cancer cell lines selected for these assays were MDA-MB-231 and MDA-MB-468, two cell lines used frequently in cancer research. Any impact on the cells' ability to pump or transport drugs (in and out) may alter the efficacy a chemotherapeutic drug, e.g., by enhancing levels of cell death) or by increasing effectiveness at lower dosage levels. The design of the breast cancer cell assays was similar to that used for bacteria in the above examples, results, and figures. In these studies, the widely used MTT (methyl thiazolyl diphenyl-tetrazolium bromide) viability assay procedure was used to assess any impact exerted by At1g74130 variant S (see, for example, the MTT procedure described by Nagaria, T. S., et al., Flavopiridol synergizes with sorafenib to induce cytotoxicity and potentiate antitumorigenic activity in EGFR/HER-2 and mutant RAS/RAF breast cancer model systems, Neoplasia, 15:939-951, 2013).

Cells were cultured at 37° C. in 96-well plates (10,000 cells in 100 ul media per well) for 24-48 hours to near confluency. After cell growth, the cells were given various treatments (different controls, with lapatinib at different dose levels, and with or without S variant At1g74130 proteins (tested 5 ug or 10 ug). The treatment volume for these MTT assays was standardized at 100 ul. The treatment volume consisted of media and combinations of control components and experimental components (media, protein buffer, lapatinib buffer, proteins and buffer, and lapatinib (10 to 30 uM)). Treatments were carried out for 24, 48, and 72 hours before the MTT assay procedure. After treatment, the wells were aspirated and replaced with solutions for the MTT assays. For the MTT assays, each well received 22 ul of MTT (2 mg/ml in PBS-sterile filtered through 0.2 um) and 78 ul complete media per well. Incubation then occurred at 37° C. for 3 hours before the addition of 100 ul of MTT stop solution[1:24 (IN HCl:isopropanol)]. The final volume was 200 ul. The assays were then read at 570 nm using a microplate scanning spectrophotometer.

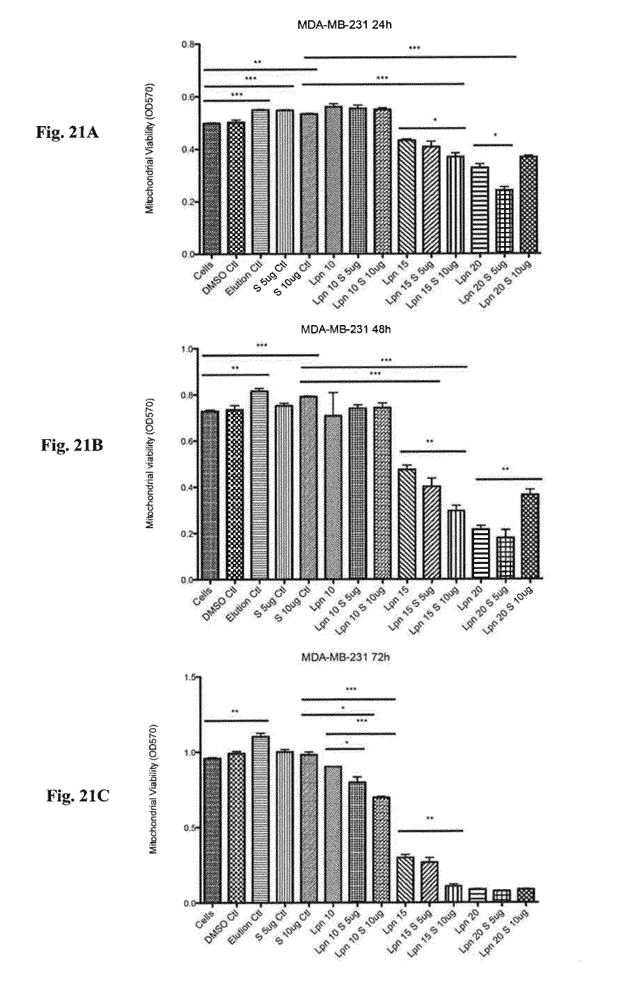

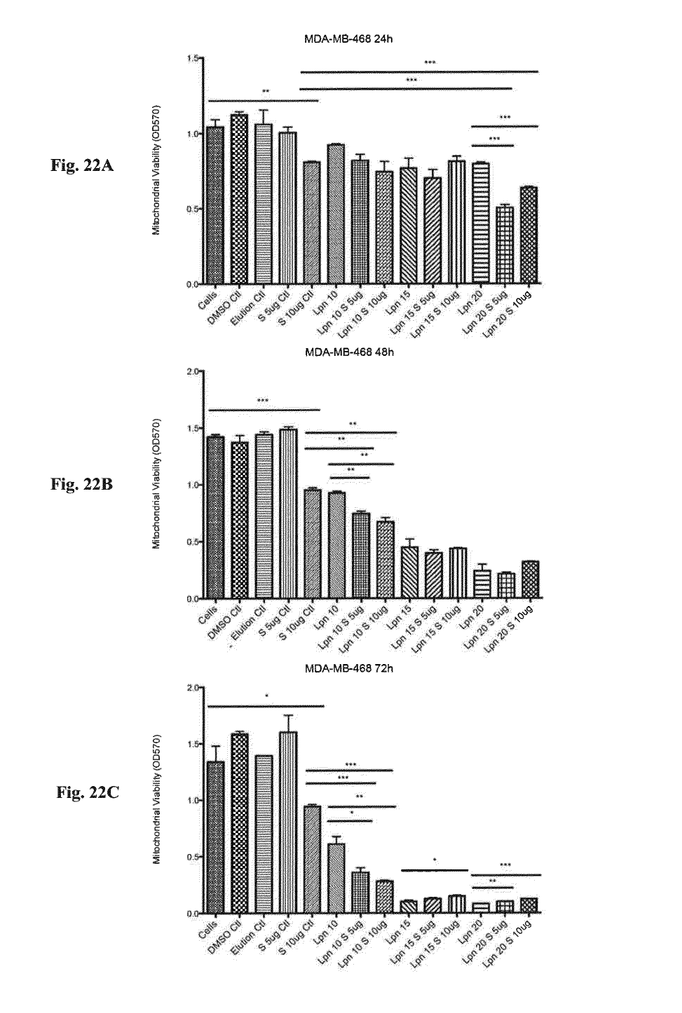

The MTT assay results are presented in FIGS. 21A, 21B, and 21C for MDA-MB-231 at 24, 48, and 72 hours, respectively, and in FIGS. 22A, 22B, and 22C for MDA-MB-468 at 24, 48, and 72 hours, respectively. The assay results are organized into two groups for all three time points and for both cell lines. The first five bars represent different types of controls in this order: 1) cells grown in media; 2) cells treated with the buffer-DMSO solution used to dissolve lapatinib; 3) cells treated with the protein elution buffer at the volume used for the protein assays; 4) cells treated with 5 ug of S variant in elution buffer; and 5) cells treated with 10 ug of protein in elution buffer. The next nine bars represent the experimental assays using different levels of lapatinib (from 10 uM to 30 uM) and S variant (5 or 10 ug). The horizontal lines between different sets of bars represent statistical significance levels (T-Test). The assays were carried out in triplicate.

Generally, the results showed that one of the main interactions in both breast cancer cell lines was conferring drug sensitivity. Both cell lines behaved in a similar manner with respect to sensitizing the cells to lower dosage levels of lapatinib. Depending on the cell line, sensitization was most prominent in the 10 or 15 uM lapatinib setting after 48 hours and as well as after 72 hours of growth after treatment. The co-treatment of lapatinib and the At1g74130 S variant increased the level of cell death by 20 to 40%. This type of response suggests a synergistic use of rhomboid variants in the delivery of therapeutic drugs, for example, co-delivery of rhomboids and drugs may increase efficacy at lower drug dosage. The application of lower drug dosage may slow down the development of drug resistance and decrease toxicity levels for patients.

### EQUIVALENTS

Those skilled in the art will recognize, or be able to ascertain using no more than routine experimentation, many equivalents to the specific embodiments of the invention described herein. Such equivalents are intended to be encompassed in the scope of the following claims.

1. A method for enhancing efficacy of an agent, comprising:
co-administering to a cell, tissue, organ, or organism the agent in a therapeutic or sub-therapeutic dose and a rhomboid protein, polypeptide, or peptide, in a suitable vehicle;
wherein the rhomboid protein is selected from At1g74130, At1g25290, and UBAC2 protein or a splice variant thereof, or the rhomboid polypeptide or peptide is derived from At1g74130, At1g25290, or UBAC2 protein or a splice variant thereof, or is a functional equivalent thereof;
wherein said co-administration enhances efficacy of the agent in the cell, tissue, organ, or organism.

2. The method of claim 1, comprising co-administering two or more agents; wherein each agent has a different biological activity or function.

3. The method of claim 1, wherein the protein or polypeptide comprises SEQ ID NO:9, SEQ ID NO:10, SEQ ID NO:11, SEQ ID NO:12, SEQ ID NO:13, or SEQ ID NO:14, or a functional equivalent thereof.

4. The method of claim 1, wherein enhancing efficacy of the agent includes treating a disease with the agent.

5. The method of claim 1, wherein enhancing efficacy of the agent includes reducing resistance to the agent in the cell, tissue, or organism.

6. The method of claim 1, wherein enhancing efficacy of the agent includes sensitizing the cell, tissue, or organism to the agent.

7. The method of claim 1, wherein enhancing efficacy of the agent includes reducing or inhibiting production of a biofilm by the cell, tissue, or organism.

8. The method of claim 1, wherein the method comprises treating antibiotic or drug resistance, infections including parasitic and fungal infections, cancerous cell growth, and progression of disease including Alzheimer's, Parkinson's, and Type-2 Diabetes.

9. A method for enhancing transport of an agent across or into a cellular membrane, comprising:
providing to a cell, tissue, organ, or organism the agent and a rhomboid protein, polypeptide, or peptide, in a suitable vehicle;
wherein the rhomboid protein is selected from At1g74130, At1g25290, and UBAC2 protein or a splice variant thereof, or the polypeptide or peptide is derived from At1g74130, At1g25290, or UBAC2 protein or a splice variant thereof, or is a functional equivalent thereof;
wherein the rhomboid protein or peptide enhances transport of the agent across or into the cellular membrane in the cell, tissue, organ, or organism.

10. The method of claim 9, wherein the protein or polypeptide comprises SEQ ID NO:9, SEQ ID NO:10, SEQ ID NO: 11, SEQ ID NO:12, SEQ ID NO:13, or SEQ ID NO:14, or a functional equivalent thereof.

11. The method of claim 9, wherein the method comprises treating antibiotic or drug resistance, infections including parasitic and fungal infections, cancerous cell growth, and progression of disease including Alzheimer's, Parkinson's, and Type-2 Diabetes.

12. A composition, comprising:
at least one agent and a rhomboid protein, polypeptide, or peptide, with a suitable vehicle;
wherein the rhomboid protein is selected from At1g74130, At1g25290, and UBAC2 protein or a splice variant thereof, or the polypeptide or peptide is derived from At1g74130, At1g25290, or UBAC2 protein or a splice variant thereof, or is a functional equivalent thereof.

13. The composition of claim 12, wherein the protein or polypeptide comprises SEQ ID NO:9, SEQ ID NO:10, SEQ ID NO:11, SEQ ID NO:12, SEQ ID NO:13, or SEQ ID NO:14, or a functional equivalent thereof.

14. The composition of claim 12, wherein the at least one agent is a therapeutic agent.

15. The composition of claim 12, wherein the composition is suitable for administration to a cell, tissue, organ, or organism.

16. The composition of claim 12, wherein the rhomboid protein, polypeptide, or peptide comprises a fusion with the suitable vehicle.

17. The composition of claim 12, wherein the rhomboid protein has an amino acid sequence with at least 80% sequence identity to SEQ ID NO:9, SEQ ID NO:10, SEQ ID NO: 11, SEQ ID NO:12, SEQ ID NO:13, or SEQ ID NO:14.

18. The composition of claim 17, wherein the protein or polypeptide comprises SEQ ID NO:9, SEQ ID NO:10, SEQ ID NO:11, SEQ ID NO:12, SEQ ID NO:13, or SEQ ID NO:14, or a functional equivalent thereof.

19. The composition of claim 12, for use in treating antibiotic or drug resistance, infections including parasitic and fungal infections, cancerous cell growth, and progression of disease including Alzheimer's, Parkinson's, and Type-2 Diabetes.

20. The composition of claim 12, for use as an antibiofilm agent.
